(19) United States
(12) Patent Application Publication
(10) Pub. No.: US 2022/0187328 A1
(43) Pub. Date: Jun. 16, 2022

(54) Methods of Terpene Profiling Cannabis Plant Material

(71) Applicant: Agriculture Victoria Services Pty Ltd, Bundoora (AU)

(72) Inventors: Aaron Christopher Elkins, Taylors Hill (AU); Simone Jane Rochfort, Reservoir (AU); Noel Cogan, Macleod (AU); German Carlos Spangenberg, Bundoora (AU); Christian Krill, Reservoir (AU)

(21) Appl. No.: 17/420,448

(22) PCT Filed: Jan. 31, 2020
(86) PCT No.: PCT/AU2020/050065

§ 371 Date: Jul. 2, 2021

(30) Foreign Application Priority Data
Jan. 31, 2019 (AU) 2019900291
Jan. 31, 2019 (AU) 2019900293
Jan. 31, 2019 (AU) 2019900294
Jan. 31, 2019 (AU) 2019900295
Jan. 31, 2019 (AU) 2019900296

(51) Int. Cl. G01N 33/94; G01N 1/40; G01N 33/00; G01N 30/72

(52) U.S. Cl. CPC G01N 33/948; G01N 1/4055; G01N 2030/025; G01N 30/7206; G01N 2001/4061; G01N 33/0098

(57) ABSTRACT

The present disclosure relates to methods of determining the terpene profile of cannabis plant material, specifically through use of gas chromatography-mass spectrometry (GC-MS). Extraction methods include liquid based extraction methods, specifically hexane based liquid extraction methods, and headspace extraction methods, specifically static headspace extraction and headspace solid phase microextraction (HS-SPME). Static headspace and HS-SPME are shown to be better for monoterpene extraction, and hexane based liquid extraction for sesquiterpene extraction.

The present application claims priority from Australian Provisional Patent Applications 2019900296, 2019900291, 2019900293, 2019900294 and 2019900295 filed on 31 Jan. 2019, the disclosures of which are hereby expressly incorporated herein by reference in their entirety.

### FIELD

The present disclosure relates generally to methods for determining the terpene profile of cannabis plant material, including uses thereof.

### BACKGROUND

Cannabis is an herbaceous flowering plant of the Cannabis genus (Rosale) that has been used for its fibre and medicinal properties for thousands of years. The medicinal qualities of cannabis have been recognised since at least 2800 BC, with use of cannabis featuring in ancient Chinese and Indian medical texts. Although use of cannabis for medicinal purposes has been known for centuries, research into the pharmacological properties of the plant has been limited due to its illegal status in most jurisdictions.

The chemistry of cannabis is varied. It is estimated that cannabis plants produce more than 400 different molecules, including phytocannabinoids, terpenes and phenolics. Cannabinoids, such as Δ-9-tetrahydrocannabinol (THC) and cannabidiol (CBD) are the most well-known and researched cannabinoids. CBD and THC are naturally present in their acidic forms, Δ-9-tetrahydrocannabinolic acid (THCA) and cannabidiolic acid (CBDA), in planta which are alternative products of a shared precursor, cannabigerolic acid (CBGA). Cannabis is often divided into categories based on the abundance of THC and CBD, in particular, Type I cannabis is THC-predominant, Type II cannabis contains both THC and CBD, and Type III is CBD-predominant.

While both the acid and corresponding neutral species of cannabinoids have been reported to have biological activity, it is the neutral forms that are more commonly associated with the effects of cannabis. The acid forms degrade naturally to the corresponding neutral forms at a slow rate via non-enzymatic processes. Typically, however, the rate of decarboxylation is increased by heating (e.g., when smoked), which liberates the neutral cannabinoid analogues to facilitate the biological activity. For example, THCA decarboxylates to its neutral form, THC, which is responsible for the psychoactive properties of cannabis. For some medicinal cannabis preparations (e.g., oil and resin preparations that are not heated for consumption), it is necessary that the cannabis material from which these preparations are derived are ‘cured’ (i.e., heated under controlled conditions) to ensure maximum decarboxylation of cannabinoids prior to consumption.

Many cannabinoids interact with the endocannabinoid system in mammals, including humans, to exert complex biological effects on the neuronal, metabolic, immune and reproductive systems. They also interact with G protein-coupled receptors (GPCRs), such as CB1 and CB2, in the human endocannabinoid system, where they are thought to play a part in the regulation of appetite, pain, mood, memory, inflammation and insulin sensitivity. Cannabinoids have also been implicated in neuronal signalling, gastrointestinal inflammation, tumorigenesis, microbial infection and diabetes.

Since different cannabinoids are likely to have different therapeutic potential, it is important to be able to screen and select for cannabis strains that have the desirable chemotypic (cannabinoid) profiles that make them suitable for medicinal use. Previous studies of the cannabinoid content of cannabis plants have largely focused on the differentiation of cannabis varieties bred for recreational or industrial use. For example, in a study conducted by Turner et al. (1979, Journal of Natural Products, 42:319-21), leaf material from 85 cannabis varieties was screened for cannabichromene (CBC), CBD and THC in order to differentiate between recreational and industrial cannabis varieties. The recreational varieties were subjected to further cannabinoid testing to identify the correct time for sampling due to the significant variation of cannabinoid biosynthesis over the life of the plan. In this context, time of sampling is important since the levels of cannabinoids vary significantly. Furthermore, any early reports of looking at CBD levels are likely to be inaccurate since CBC had been previously been misidentified as CBD. More recently, nuclear magnetic resonance (NMR) spectroscopy and RT-PCR analysis has been used to investigate the metabolome and cannabinoid biosynthesis in the trichomes of Cannabis sativa “Bebiol” in the last four weeks (i.e., week five to week nine) of the flowering period (Happyana and Kayser, 2016, Planta Medica, 82:1217-23). In this study, cannabinoid biosynthesis increased in week five to six but was relatively static in the later weeks once the buds were mature. Following biosynthesis, there is a slow decline in certain cannabinoids, particularly THC as the plant material ages.

In addition to cannabinoids, Cannabis also produces an additional class of biologically active molecules, the volatile terpenes. These molecules are responsible for the distinct aroma and “flavor” profile of cannabis strains. Terpenes found in cannabis are also found in other plant species and have a range of bioactivities associated with them. For example, D-limonene, exhibits potent anti-cancer, anxiolytic and immunostimulating properties, β-myrcene, is a potent anti-inflammatory, analgesic, and anxiolytic component, α-pinene is an acetylcholinesteral inhibitor, linalool possesses analgesic, anti-anxiety, anti-inflammatory, and anticonvulsant activities and β-caryophyllene possesses anti-inflammatory and gastric cytoprotector activities (Andre et al. 2016, Frountiers in Plant Science, 7, Article 19: 1-17). Terpenes are also commonly thought to contribute to the “entourage effect” associated with cannabis as described in, for example, Russo, 2011, British Journal of Pharmacology, 163(7): 1344-1364.

Since different terpenes have different therapeutic potential and alter the way patients perceive medicinal cannabis, it is advantageous to select for new cannabis varieties that have a terpene profile enriched for specific terpenes suitable for therapeutic use.

Despite recent advances in the analysis of cannabinoids, there has been lack of sufficient systematic analysis of terpenes for the purpose of precision breeding of cannabis plants for medicinal use. There remains, therefore, an urgent need for improved tools and methods for measuring terpenes in plant material, and in a manner that is suitable for use in the systematic breeding and selection of improved cannabis varieties comprising a terpene profile enriched for specific terpenes that make them suitable for therapeutic use.

### SUMMARY

In an aspect disclosed herein, there is provided a method for determining a monoterpene profile of cannabis plant material, the method comprising:
a. obtaining a reference spectrum of known monoterpenes;
b. selectively extracting one or more monoterpenes from the cannabis plant material;
c. obtaining spectroscopic data from the monoterpenes separated in step (b);
d. comparing the spectroscopic data to the reference spectrum to identify and/or measure the level of one or more monoterpenes to determine the monoterpene profile of the cannabis plant material.

In another aspect disclosed herein, there is provided a method for detecting a sesquiterpene profile of cannabis plant material, the method comprising:
a. obtaining a reference spectrum of known sesquiterpenes;
b. selectively extracting one or more sesquiterpenes from the cannabis plant material;
c. obtaining spectroscopic data from the sesquiterpenes separated in step (b);
d. comparing the spectroscopic data to the reference spectrum to identify and/or measure the level of one or more sesquiterpenes to determine the sesquiterpene profile of the cannabis plant material.

In another aspect disclosed herein, there is provided a method for selecting a cannabis plant comprising a terpene profile enriched for monoterpenes, the method comprising:
a. harvesting plant material from a plurality of different cannabis plants;
b. at least partially drying the harvested plant material of step (a);
c. measuring in the at least partially dried plant material of step (b) a level of one or more monoterpenes and to generate a monoterpene profile for each of the plurality of cannabis plants in accordance with the method as disclosed herein; and
d. on the basis of the measurements from step (c), selecting from the plurality of different cannabis plants a cannabis plant comprising a terpene profile that is enriched for monoterpenes.

In another aspect disclosed herein, there is provided a method for selecting a cannabis plant comprising a terpene profile enriched for sesquiterpenes, the method comprising:
a. harvesting plant material from a plurality of different cannabis plants;
b. at least partially drying the harvested plant material of step (a);
c. measuring in the at least partially dried plant material of step (b) a level of one or more sesquiterpenes and to generate a sesquiterpene profile for each of the plurality of cannabis plants in accordance with the method as disclosed herein; and
d. on the basis of the measurements from step (c), selecting from the plurality of different cannabis plants a cannabis plant comprising a terpene profile that is enriched for sesquiterpene.

### BRIEF DESCRIPTION OF THE FIGURES

FIG. 1 shows an analysis of a cannabis composite sample using three different extraction methods. (A) A graphical representation of Counts vs Acquisition Time (min) (x-axis) against peak height (y-axis) following static headspace. (B) A graphical representation of Counts vs Acquisition Time (min) (x-axis) against peak height (y-axis) following solid phase micro-extraction (SPME). (C) A graphical representation of Counts vs Acquisition Time (min) (x-axis) against peak height (y-axis) following hexane-based liquid extraction.

FIG. 2 shows the distribution of terpene content in cannabis plants. (A) Principal component analysis (PCA) of terpene content across cannabis plants, PCA Scores on PC1 (x-axis; 69.48%) against PCA Scores on PC2 (y-axis; 16.62%). (B) Loading plot (PC1) demonstrating that myrcene, α-pinene and limonene are in higher abundance (y-axis; 69.48%) against variable (x-axis).

FIG. 3 shows the relative abundance (y-axis; peak area) of (A) β-pinene and (B) myrcene in different cannabis plants.

FIG. 4 shows the correlation between cannabinoid and terpene content in cannabis plants. Areas with strong negative correlation are highlighted by the solid boxes. Areas of strong positive correlation are highlighted in the dashed boxes.

FIG. 5 shows the terpene profile of a cannabis plant comprising a cannabinoid profile enriched for total CBD. A graphical representation of the terpene content (x-axis) against peak area (y-axis; counts) for the Cannabis-1 strain.

FIG. 6 shows the terpene profile of cannabis plants comprising a cannabinoid profile enriched for total CBD and for total THC (CBD:THC). (A-G) Graphical representations of the terpene content (terpene; x-axis) against peak area (counts; y-axis) for the Cannabis-2, Cannabis-3, Cannabis-6 and Cannabis-7, Cannabis-8, Cannabis-9, Cannabis-10, Cannabis-11, Cannabis-12, Cannabis-13, Cannabis-14, Cannabis-15, Cannabis-16, Cannabis-17, Cannabis-18, Cannabis-19, Cannabis-20, Cannabis-21 the Cannabis-22, Cannabis-23, Cannabis-24. Cannabis-25, Cannabis-26, Cannabis-27, Cannabis-28, Cannabis-29 and Cannabis-30 strains.

FIG. 7 shows the terpene profile of cannabis plants comprising a cannabinoid profile enriched for total THC and total CBG. (A-F) Graphical representations of the terpene content (terpene; x-axis) against peak area (counts; y-axis) for the Cannabis-31, Cannabis-32, Cannabis-34, Cannabis-35, Cannabis-36, Cannabis-37, Cannabis-38, Cannabis-39, Cannabis-40, Cannabis-41, Cannabis-42, Cannabis-43, Cannabis-44, Cannabis-45, Cannabis-46, Cannabis-47, Cannabis-48. Cannabis-49, Cannabis-50, Cannabis-51, Cannabis-52, Cannabis-53, Cannabis-54, Cannabis-55, Cannabis-56, Cannabis-57, Cannabis-58 and Cannabis-59 strains.

FIG. 8 shows the terpene profile of cannabis plant comprising a cannabinoid profile enriched for total THC, total CBG and total THCV. (A-B) Graphical representations of the terpene content (terpene; x-axis) against peak area (counts; y-axis) for Cannabis-60, Cannabis-61, Cannabis-62, Cannabis-63, Cannabis-64, Cannabis-65, Cannabis-66, Cannabis-67, Cannabis-68, Cannabis-69, Cannabis-70 and Cannabis-71 strains.

FIG. 9 shows the comparison of peak shapes for early eluting compounds using three different injection solvents: hexane (grey dotted line), ethyl acetate (black solid line) and isopropanol (black dashed line).

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor). Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen. N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “an inflorescence” includes a single inflorescence, as well as two or more inflorescences; and so forth.

The present disclosure is predicated, at least in part, on the unexpected finding monoterpenes and sesquiterpenes may be selectively extracted and analysed to generate monoterpene and/or sesquiterpene profiles from cannabis plant material.

### Cannabis

As used herein, the term “cannabis plant” means a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of Cannabis are morphologically variable, which has resulted in difficulty defining the taxonomic organisation of the genus. In an embodiment, the cannabis plant is C. sativa.

The terms “plant”, “cultivar”, “variety”, “strain” or “race” are used interchangeably herein to refer to a plant or a group of similar plants according to their structural features and performance (i.e., morphological and physiological characteristics).

The reference genome for C. sativa is the assembled draft genome and transcriptome of “Purple Kush” or “PK” (van Bakal et al. 2011, Genome Biology, 12: R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

As used herein, the term “plant part” refers to any part of the plant, illustrative examples of which include an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. The term “plant part” also includes any material listed in the Plant Part Code Table as approved by the Australian Therapeutic Goods Administration (TGA) Business Services (TBS). In an embodiment, the part is selected from the group consisting of an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. In a preferred embodiment, the part is a cannabis bud.

### Cannabinoids

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated forms thereof.

TABLE 1
Cannabinoids and their properties.

| | | Chemical |
|---|---|---|
| | | properties/ |
| | | [M + H]⁺ ESI |
| Name | Structure | MS |
| Δ9-tetrahydrocannabinol (THC) | 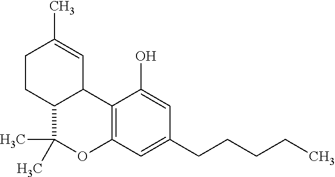 | Psychoactive, decarboxylation product of THCA m/z 315.2319 |
| Δ9- tetrahydrocannabinolic acid (THCA/THCA-A) | 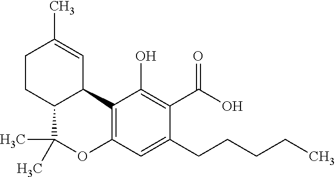 [SMILES: CCCCCc1cc2c(c(O)c1C(=O)O)[C@@H]1C=C(C)CC[C@H]1C(C)(C)O2] | m/z 359.2217 |
| cannabidiol (CBD) | 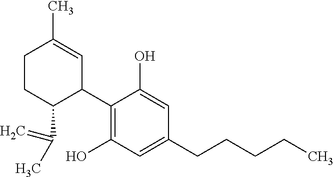 [SMILES: C=C(C)[C@@H]1CCC(C)=CC1c1c(O)cc(CCCCC)cc1O] | decarboxylation product of CBDA m/z 315.2319 |
| cannabidiolic acid (CBDA) | 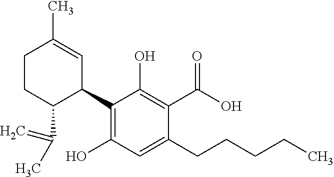 [SMILES: C=C(C)[C@@H]1CCC(C)=C[C@H]1c1c(O)cc(CCCCC)c(C(=O)O)c1O] | m/z 359.2217 |
| cannabigerol (CBG) | 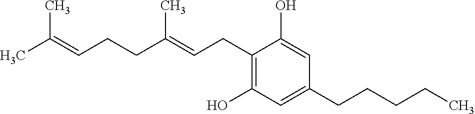 [SMILES: CCCCCc1cc(O)c(C/C=C(\C)CCC=C(C)C)c(O)c1] | Non- intoxicating, decarboxylation product of CBGA m/z 317.2475 |
| cannabigerolic acid (CBGA) | 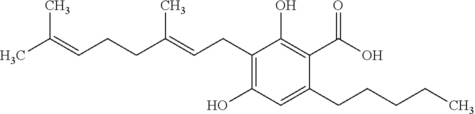 [SMILES: CCCCCc1cc(O)c(C/C=C(\C)CCC=C(C)C)c(O)c1C(=O)O] | m/z 361.2373 |
| cannabichromene (CBC) | 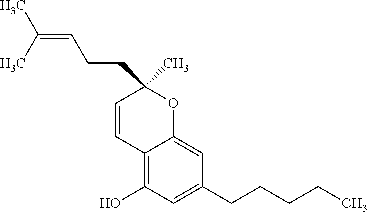 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 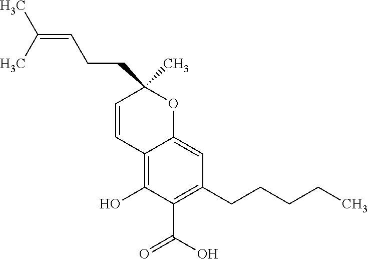 [SMILES: CCCCCc1cc2c(c(O)c1C(=O)O)C=C[C@@](C)(CCC=C(C)C)O2] | m/z 359.2217 |
| cannabicyclol (CBL) | 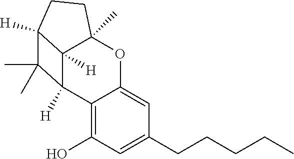 | Non- psychoactive, 16 isomers known. Derived from non-enzymatic conversion of CBC m/z 315.2319 |
| cannabinol (CBN) | 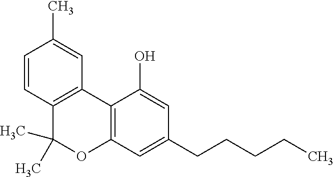 [SMILES: CCCCCc1cc(O)c2c(c1)OC(C)(C)c1ccc(C)cc1-2] | Likely degradation product of THC m/z 311.2006 |
| cannabinolic acid (CBNA) | 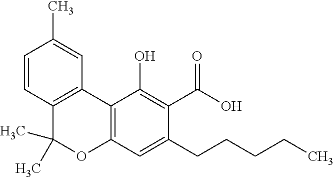 [SMILES: CCCCCc1cc2c(c(O)c1C(=O)O)-c1cc(C)ccc1C(C)(C)O2] | m/z 355.1904 |
| tetrahydrocannabivarin (THCV) | 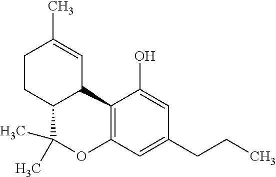 | decarboxylation product of THCVA m/z 287.2006 |
| tetrahydrocannabivarinic acid (THCVA) | 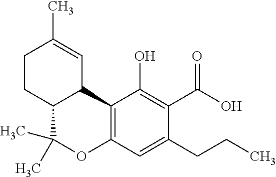 | m/z 331.1904 |
| cannabidivarin (CBDV) | 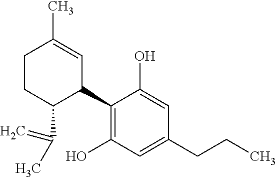 | m/z 287.2006 |
| cannabidivarinic acid (CBDVA) | 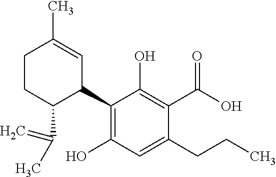 | m/z 331.1904 |
| Δ8-tetrahydrocannabinol (d8-THC) | 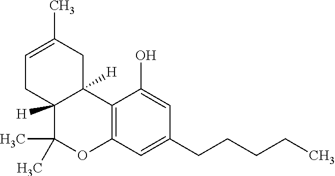 [SMILES: CCCCCc1cc(O)c2c(c1)OC(C)(C)[C@@H]1CC=C(C)C[C@@H]21] | m/z 315.2319 |

Cannabinoids are synthesised in cannabis plants as carboxylic acids. While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying, heating and/or curing (i.e., heating for a specific time and temperature to ensure maximum decarboxylation) the plant material. Persons skilled in the art would be familiar with methods by which decarboxylation of cannabinoids can be promoted, illustrative examples of which include combustion, vaporisation, curing, drying, heating and baking.

“Δ-9-tetrahydrocannabinolic acid” or “THCA-A” is synthesised from the CBGA precursor by THCA synthase. The neutral form “Δ-9-tetrahydrocannabinol” or “THC” is associated with psychoactive effects of cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

“Cannabidiolic acid” or “CBDA” is also a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

### Cannabinoid Profile

The term “cannabinoid profile” as used herein refers to a representation of the type, amount, level, ratio and/or proportion of cannabinoids that are present in the cannabis plant or part thereof, as typically measured within plant material derived from the plant or plant part, including an extract therefrom.

The term “enriched” is used herein to refer to a selectively higher level of one or more cannabinoids or terpenes in the cannabis plant or part thereof. For example, a cannabinoid profile enriched for total CBD refers to plant material in which the amount of total CBD (total CBD and/or total CBDA) is greater than the amount of any of the other cannabinoids that may also be present (including constitutively present) in the plant material.

The cannabinoid profile in a cannabis plant will typically predominantly comprise the acidic form of the cannabinoids, but may also comprise some decarboxylated (neutral) forms thereof, at various concentrations or levels at any given time (i.e., at propagation, growth, harvest, drying, curing, etc.). Thus, the term “total cannabinoid” is used herein to refer to the decarboxylated and/or acid form of said cannabinoid. For example, “total CBD” refers to total CBD and/or total CBDA, “total THC” refers to total THC and/or total THCA, “total CBC” refers to CBC and/or CBCA, “total CBG” refers to CBG and/or CBGA, “total CBN” refers to total CBN and/or total CBNA, “total THCV” refers to total THCV and/or total THCVA, “total CBDV” refers to total CBDV and/or total CBDVA, and so forth.

The terms “level”, “content”, “concentration” and the like, are used interchangeably herein to describe an amount of the referenced compound, and may be represented in absolute terms (e.g., mg/g, mg/ml, etc) or in relative terms, such as a ratio to any or all of the other compounds in the cannabis plant material or as a percentage of the amount (e.g., by weight) of any or all of the other compounds (i.e., a reference cannabinoid) in the cannabis plant material.

As used herein, the term “plant material” is to be understood to mean any part of the cannabis plant, including the leaves, stems, roots, and inflorescence, or parts thereof, as described elsewhere herein, as well as extracts, illustrative examples of which include kief or hash, which includes trichomes and glands. In an embodiment, the plant material is derived from a female cannabis plant. In another embodiment, the plant material is an inflorescence or a leaf. In a preferred embodiment, the plant material is an inflorescence.

In an embodiment, the cannabis plant material is at least partially dried.

The term “drying” as used herein refers to any method for drying the plant material. Illustrative examples include air-drying, curing, and heat drying. In an embodiment, the plant material is dried in a temperature, light and humidity controlled environment, such as a temperature of about 21° C. and a humidity of from about 38% and 45% RH. In another embodiment, heat is applied to the plant material during the drying process to cure the dried plant material. Temperatures suitable for curing dried plant material would be known to persons skilled in the art, illustrative examples of which include a temperature from about 60° C. to about 225° C., preferably from about 100° C. to about 150° C., preferably from about 110° C. to about 130° C., or more preferably about 120° C. In an embodiment, the dried plant material is cured by heating the dried plant material at about 120° C. for 2 hours.

It is to be understood that the term “dry”, “drying” and the like is not intended to mean the absence of moisture in the plant material, and therefore includes any state in which at least some moisture has been removed from the plant material. Persons skilled in the art will be familiar with the extent to which cannabis plant material can be dried to allow for extraction of the desirable compound(s), including decarboxylated cannabinoids. In an embodiment, the harvested plant material is dried under conditions and for a period of time that gives rise to a loss of at least 5%, preferably at least 10%, preferably at least 20%, preferably at least 30%, preferably at least 40%, preferably at least 50%, preferably at least 60%, preferably at least 70%, preferably at least 80%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% of the moisture content of the plant material at the time of harvest.

The term “inflorescence” as used herein means the complete flower head of the cannabis plant, comprising stems, stalks, bracts, flowers and trichomes (i.e., glandular, sessile and stalked trichomes). In a preferred embodiment, the plant material comprises cannabis trichomes.

As noted elsewhere herein, cannabinoids are synthesised in cannabis plants predominantly in acid form (i.e., as carboxylic acids). While some decarboxylation may occur in the plant, decarboxylation typically occurs post-harvest and is increased by exposing the plant material to heat. Thus, in an embodiment, the methods disclosed herein comprise obtaining spectroscopic data from plant material that has not been heat treated under conditions and for a period of time that would otherwise result in the decarboxylation of acid forms of cannabinoids in the plant material.

In an embodiment, the cannabis plant material has a predetermined cannabinoid profile that is selected from the group consisting of (i) a cluster 1 cannabinoid profile; (ii) a cluster 2 cannabinoid profile; (iii) a cluster 3 cannabinoid profile; and (iv) a cluster 4 cannabinoid profile.

### Cluster 1 Cannabinoid Profile

The term “cluster 1 cannabinoid profile” as used herein refers to a cannabinoid profile that is characterised by a level of total CBD in the plant material that is greater than the level of total THC. Accordingly, the cannabis plant of the invention may be variously described as “high-CBD”, “CBD-enriched” or “high-CBD, low-THC”. Those skilled in the art would understand this terminology to mean a cannabis plant that produced higher levels of CBD and/or CBDA relative to the level of THC and/or THCA.

In an embodiment, the level of total CBD is at least about 80% by weight of the total cannabinoid content of the dry weight of the plant material, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material.

In an embodiment, the level of total THC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material.

In an embodiment, the level of total CBD and the level of total THC are present at a ratio of from about 10:1 to about 50:1, preferably from about 10:1 to about 40:1, preferably from about 10:1 to about 30:1, preferably from about 15:1 to about 50:1, preferably from about 15:1 to about 40:1, preferably from about 15:1 to about 30:1, preferably from about 20:1 to about 50:1, preferably from about 20:1 to about 40:1, or more preferably from about 20:1 to about 30:1 (CBD:THC) in the plant material.

In an embodiment, the reference cannabinoid is total CBC. In another embodiment, the level of total CBD is present at a ratio of from about from about 10:1 to about 50:1 to the level of total CBC, preferably from about 10:1 to about 40:1, preferably from about 10:1 to about 30:1, preferably from about 15:1 to about 50:1, preferably from about 15:1 to about 40:1, preferably from about 15:1 to about 30:1, preferably from about 20:1 to about 50:1, preferably from about 20:1 to about 40:1, or more preferably from about 20:1 to about 30:1 (CBD:CBC) in the plant material.

In another embodiment, the level of total CBC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material.

In an embodiment, the reference cannabinoid is total CBG. In another embodiment, the level of total CBD is present at a ratio of from about from about 10:1 to about 100:1 to the level of total CBG, preferably from about 10:1 to about 90:1, preferably from about 10:1 to about 80:1, preferably from about 20:1 to about 100:1, preferably from about 20:1 to about 90:1, preferably from about 20:1 to about 80:1, preferably from about 30:1 to about 100:1, preferably from about 30:1 to about 90:1, preferably from about 30:1 to about 80:1, preferably from about 40:1 to about 100:1, preferably from about 40:1 to about 90:1, preferably from about 40:1 to about 80:1, preferably from about 50:1 to about 100:1, preferably from about 50:1 to about 90:1, preferably from about 50:1 to about 80:1, preferably from about 60:1 to about 100:1, preferably from about 60:1 to about 90:1, preferably from about 60:1 to about 80:1, preferably from about 70:1 to about 100:1, preferably from about 70:1 to about 90:1, or more preferably from about 70:1 to about 80:1 (CBD:CBG) in the plant material.

In another embodiment, the level of total CBG is from about 1% and 5%, preferably from about 1% and 4%, preferably from about 1% and 3%, or more preferably from about 1% and 2% by weight of the total cannabinoid content of the dry weight of the plant material.

In an embodiment, the reference cannabinoid is total CBN. In another embodiment, the level of total CBD is present at a ratio of from about from about 2000:1 to about 3000:1 of the level of total CBN, preferably from about 2000:1 to about 3000:1, preferably from about 2100:1 to about 3000:1, preferably from about 2200:1 to about 3000:1, preferably from about 2300:1 to about 3000:1, preferably from about 2400:1 to about 3000:1, or more preferably from about 2500:1 to about 3000:1 (CBD:CBN) in the plant material.

In another embodiment, the level of total CBN is from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material.

In an embodiment, the reference cannabinoid is total CBDV. In another embodiment, the level of total CBD is present at a ratio of is from about 10:1 to about 80:1 to the level of total CBDV, preferably from about 10:1 to about 70:1, preferably from about 20:1 to about 80:1, preferably from about 20:1 to about 70:1, preferably from about 30:1 to about 80:1, preferably from about 30:1 to about 70:1, preferably from about 40:1 to about 80:1, preferably from about 40:1 to about 70:1, preferably from about 50:1 to about 80:1, preferably from about 50:1 to about 70:1, preferably from about 60:1 to about 80:1, or more preferably from about 60:1 to about 70:1 (CBD:CBDV) in the plant material.

In another embodiment, the level of total CBDV in the plant material is from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of the plant material.

In an embodiment, the reference cannabinoid is total THCV. In another embodiment, the level of total CBD is present at a ratio of from about 400:1 to about 700:1 of the level of total THCV, preferably from about 400:1 to about 600:1, preferably from about 500:1 to about 700:1, or more preferably from about 500:1 to about 600:1 (CBD:THCV) in the plant material.

In another embodiment, the level of total THCV is from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material; and
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material,
(iii) optionally a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material;
(iv) optionally a level of total CBG of from about 1% and 5%, preferably from about 1% and 4%, preferably from about 1% and 3%, or more preferably from about 1% and 2% by weight of the total cannabinoid content of the dry weight of the plant material;
(v) optionally a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material;
(vi) optionally a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of the plant material; and
(vii) optionally a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material; and
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material; and
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material; and
(iv) a level of total CBG of from about 1% and 5%, preferably from about 1% and 4%, preferably from about 1% and 3%, or more preferably from about 1% and 2% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material;
(v) a level of total CBG of from about 1% and 5%, preferably from about 1% and 4%, preferably from about 1% and 3%, or more preferably from about 1% and 2% by weight of the total cannabinoid content of the dry weight of the plant material; and
(iv) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material;
(iv) a level of total CBG of from about 1% and 5%, preferably from about 1% and 4%, preferably from about 1% and 3%, or more preferably from about 1% and 2% by weight of the total cannabinoid content of the dry weight of the plant material;
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material; and
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material;
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material;
(iv) a level of total CBG of from about 1% and 5%, preferably from about 1% and 4%, preferably from about 1% and 3%, or more preferably from about 1% and 2% by weight of the total cannabinoid content of the dry weight of the plant material;
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material;
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of the plant material; and
(vii) a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of the plant material.

In an embodiment, the cannabis plant comprises:
(i) a level of total CBD of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of the plant material; and/or
(ii) a level of total THC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, preferably from about 2% to about 5%, preferably from about 3% to about 10%, preferably from about 3% to about 9%, preferably from about 3% to about 8%, preferably from about 3% to about 7%, preferably from about 3% to about 6%, or more preferably from about 3% to about 5% by weight of the total cannabinoid content of the dry weight of the plant material; and/or
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of the plant material; and/or
(iv) a level of total CBG of from about 1% and 5%, preferably from about 1% and 4%, preferably from about 1% and 3%, or more preferably from about 1% and 2% by weight of the total cannabinoid content of the dry weight of the plant material; and/or
(v) a level of total CBN of from about 0.01% to about 0.1%, preferably from about 0.01% to about 0.09%, preferably from about 0.01% to about 0.08%, preferably from about 0.01% to about 0.07%, preferably from about 0.01% to about 0.06%, or more preferably from about 0.01% to about 0.05% by weight of the total cannabinoid content of the dry weight of the plant material; and/or
(vi) a level of total CBDV of from about 1% to about 10%, preferably from about 1% to about 9%, preferably from about 1% to about 8%, preferably from about 1% to about 7%, preferably from about 1% to about 6%, or more preferably from about 1% to about 5% by weight of the total cannabinoid content of the of dry weight of the plant material; and/or
(vii) a level of total THCV of from about 0.05% to about 1%, preferably from about 0.05% to about 0.09%, preferably from about 0.05% to about 0.08%, preferably from about 0.05% to about 0.07%, preferably from about 0.05% to about 0.06%, preferably from about 0.05% to about 0.04%, preferably from about 0.05% to about 0.03%, or more preferably from about 0.05% to about 0.02% by weight of the total cannabinoid content of the dry weight of the plant material.

### Cluster 2 Cannabinoid Profile

The term “cluster 2 cannabinoid profile” as used herein refers to a cannabinoid profile that is characterised by an approximately equal level of total CBD and THC in the plant material, which is greater than the level of other minor cannabinoids. Accordingly, the cannabis plant of the invention may be variously described as “high-CBD and -THC”, “CBD- and THC-enriched” or “high-CBD and -THC”. Those skilled in the art would understand this terminology to mean a cannabis plant that produced higher levels of CBD and/or CBDA and THC and/or THCA, relative to the level of other minor cannabinoids.

In an embodiment, the level of total CBD is at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material.

In an embodiment, the level of total THC is at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material.

In an embodiment, total CBD and total THC are present in a ratio of from about 1:1 to about 5:1, preferably from about 1:1 to about 4:1, or more preferably from about 1:1 to about 3:1 (CBD:THC). In another embodiment, total CBD and total THC are present in a ratio of about 1:1.

In an embodiment, the reference cannabinoid is total CBC. In another embodiment, total CBD and total THC (CBD+THC) is present at a ratio of from about 10:1 to about 50:1 to the level of total CBC, preferably from about 10:1 to about 49:1, preferably from about 10:1 to about 48:1, preferably from about 10:1 to about 47:1, preferably from about 10:1 to about 46:1, preferably from about 10:1 to about 45:1, preferably from about 10:1 to about 44:1, preferably from about 10:1 to about 43:1, preferably from about 10:1 to about 42:1, preferably from about 10:1 to about 41:1, or more preferably from about 10:1 to about 40:1 (CBD+THC:CBC).

In another embodiment, the level of total CBC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material.

In an embodiment, the reference cannabinoid is total CBG. In another embodiment, CBD+THC is present at a ratio of from about 10:1 to about 110:1 to the level of total CBG, preferably from about 20:1 to about 110:1, preferably from about 10:1 to about 110:1, preferably from about 30:1 to about 110:1, preferably from about 40:1 to about 110:1, preferably from about 50:1 to about 110:1, preferably from about 60:1 to about 110:1, preferably from about 70:1 to about 110:1, preferably from about 80:1 to about 110:1, preferably from about 90:1 to about 110:1, or more preferably from about 100:1 to about 110:1 (CBD+THC:CBG).

In another embodiment, the level of total CBG is from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material.

In an embodiment, the reference cannabinoid is total CBN. In another embodiment, CBD+THC is present at a ratio of from about 400:1 to about 4000:1 to the level of total CBN, preferably from about 400:1 to about 3900:1, preferably from about 400:1 to about 3800:1, preferably from about 400:1 to about 3700:1, preferably from about 400:1 to about 3600:1, preferably from about 400:1 to about 3500:1, preferably from about 400:1 to about 3400:1, preferably from about 400:1 to about 3300:1, preferably from about 400:1 to about 3200:1, preferably from about 400:1 to about 3100:1, or more preferably from about 400:1 to about 3000:1 (CBD+THC:CBG).

In another embodiment, the level of total CBN is from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material.

In an embodiment, the reference cannabinoid is total CBDV. In another embodiment, CBD+THC is present at a ratio of from about 100:1 to about 2000:1 to the level of total CBDV, preferably from about 100:1 to about 1900:1, preferably from about 100:1 to about 1800:1, preferably from about 100:1 to about 1700:1, preferably from about 100:1 to about 1600:1, preferably from about 100:1 to about 1500:1, preferably from about 100:1 to about 1400:1, preferably from about 100:1 to about 1300:1, preferably from about 100:1 to about 1200:1, preferably from about 100:1 to about 1100:1, or more preferably from about 100:1 to about 1000:1 (CBD+THC:CBDV).

In another embodiment, the level of total CBDV is from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material.

In an embodiment, the reference cannabinoid is total THCV. In another embodiment, CBD+THC is present at a ratio of from about 100:1 to about 600:1 to the level of total THCV, preferably from about 100:1 to about 590:1, preferably from about 100:1 to about 580:1, preferably from about 100:1 to about 570:1, preferably from about 100:1 to about 560:1, preferably from about 100:1 to about 550:1, preferably from about 100:1 to about 540:1, preferably from about 100:1 to about 530:1, preferably from about 100:1 to about 520:1, preferably from about 100:1 to about 510:1, or more preferably from about 100:1 to about 500:1 (CBD+THC:THCV).

In another embodiment, the level of total THCV is from about 0.01% to about 1%, preferably from about 0.02% to about 1%, preferably from about 0.03% to about 0.1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC is at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) optionally a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) optionally a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material;
(v) optionally a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material;
(vi) optionally a level of total CBDV of from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material; and
(vii) optionally a level of total THCV of from about 0.01% to about 1%, preferably from about 0.02% to about 1%, preferably from about 0.03% to about 0.1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material; and
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material; and
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material; and
(iv) a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material; and
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material:
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material; and
(vi) a level of total CBDV of from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material;
(iv) a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material;
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material;
(vi) a level of total CBDV of from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material; and
(vii) a level of total THCV of from about 0.01% to about 1%, preferably from about 0.02% to about 1%, preferably from about 0.03% to about 0.1%, preferably from about 0.04% to about 1%, preferably from about 0.05% to about 1%, preferably from about 0.06% to about 1%, preferably from about 0.07% to about 1%, preferably from about 0.08%, to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total CBD of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(ii) a level of total THC of at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material;
(iii) a level of total CBC of from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material; and/or
(iv) a level total CBG of from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material; and/or
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material; and/or
(vi) a level of total CBDV of from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material; and/or
(vii) a level of total THCV of from about 0.01% to about 1%, preferably from about 0.02% to about 1%, preferably from about 0.03% to about 0.1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

### Cluster 3 Cannabinoid Profile

The term “cluster 3 cannabinoid profile” as used herein refers to a cannabinoid profile that is characterised by high levels of total THC, but is also relatively high levels of total CBG. Accordingly, the cannabis plant of the present disclosure may be variously described as “high THC/high CBG”, “high THC/CBG”, “THC/CBD-enriched” and the like. Those skilled in the art would understand this terminology to mean a cannabis plant that produced higher levels of THC and/or THCA and CBG and/or CBGA relative to the level of other cannabinoids, such as CBD and/or CBDA.

“Cannabigerolic acid” or “CBGA” is the common precursor of both THCA and cannabidiolic acid” or “CBDA”. Its neutral form, “cannabigerol” or “CBG” has relatively weak agonistic effect on the CB1 and CB2 receptors. However, CBG acts as an AEA uptake inhibitor, α-2 adrenoceptor agonist and a moderate 5-HT1A antagonist. As a result, CBG has been suggested for use as a sedative, anti-inflammatory, anti-anxiety, anti-nausea, atypical anti-psychotic, anti-fungal and as a cancer treatment.

In an embodiment, the level of total THC is at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material.

In an embodiment, the level of total CBG is at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material.

In an embodiment, total THC and total CBG are present in a ratio of from about 5:1 to about 50:1, preferably from about 6:1 to about 50:1, preferably from about 7:1 to about 50:1, preferably from about 8:1 to about 50:1, preferably from about 9:10 to about 50:1, or more preferably from about 10:1 to about 50:1 (THC:CBG).

In an embodiment, the reference cannabinoid is total CBD. In another embodiment, total THC and total CBG (THC+CBG) are present in a ratio of from about 50:1 to about 500:1 to the level of total CBD, preferably from about 50:1 to about 490:1, preferably from about 50:1 to about 480:1, preferably from about 50:1 to about 470:1, preferably from about 50:1 to about 460:1, preferably from about 50:1 to about 450:1, preferably from about 50:1 to about 440:1, preferably from about 50:1 to about 430:1, preferably from about 50:1 to about 420:1, preferably from about 50:1 to about 410:1, preferably about 50:1 to about 400:1, preferably from about 60:1 to 500:1, preferably from about 70:1 to 500:1, preferably from about 80:1 to 500:1, preferably from about 90:1 to about 500:1, or more preferably from about 100:1 to about 400:1 (THC+CBG:CBD).

In another embodiment, the level of total CBD is from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material.

In an embodiment, the reference cannabinoid is total CBC. In another embodiment, THC+CBG are present in a ratio of from about 10:1 to about 120:1 to the level of total CBC, preferably from about 11:1 to about 120:1, preferably from about 12:1 to about 120:1, preferably from about 13:1 to about 120:1, preferably from about 14:1 to about 120:1, preferably from about 15:1 to about 120:1, preferably from about 10:1 to about 119:1, preferably from about 10:1 to about 118:1, preferably from about 10:1 to about 117:1, preferably from about 10:1 to about 116:1, preferably from about 10:1 to about 115:1, or more preferably from about 15:1 to about 115:1 (CBD+THC:CBC).

In another embodiment, the level of total CBC is from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material.

In an embodiment, the reference cannabinoid is total CBN. In another embodiment, THC+CBG are present in a ratio of from about 200:1 to about 1500:1 to the level of total CBN, preferably from about 200:1 to about 1400:1, or more preferably from about 200:1 to about 1300:1 (CBG+THC:CBN).

In another embodiment, the level of total CBN is from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material.

In an embodiment, the reference cannabinoid is total THCV. In another embodiment, THC+CBG are present in a ratio of from about 50:1 to about 700:1 to the level of total THCV, preferably from about 60:1 to about 700:1, preferably from about 70:1 to about 700:1, preferably from about 80:1 to about 700:1, preferably from about 90:1 to about 700:1, preferably from about 50:1 to about 650:1, preferably from about 50:1 to about 600:1, preferably from about 50:1 to about 550:1, or more preferably from about 90:1 to about 550:1 (CBD+THC:THCV).

In another embodiment, the level of total THCV is from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabinoid profile does not include total CBDV, wherein total CBDV comprises cannabidivarin (CBDV) and/or cannabidivarinic acid (CBDVA).

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and
(ii) a level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material;
(iii) optionally a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material;
(iv) optionally a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material;
(v) optionally a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material; and
(vi) optionally a level of total THCV of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material; and
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material;
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material; and
(iv) a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material:
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material;
(iv) a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material; and
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material;
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material;
(vi) a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material; and
(iv) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material;

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material;
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material;
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material;
(iv) a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material;
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material; and
(vi) a level of total THCV of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total THC of at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% by weight of the total cannabinoid content of the dry weight of plant material; and/or
(ii) the level of total CBG of at least 1%, preferably at least 2%, preferably at least 3%, preferably at least 4%, preferably at least 5%, preferably at least 6%, preferably at least 7%, preferably at least 9%, or more preferably at least 10% by weight of the total cannabinoid content of the dry weight of plant material; and/or
(iii) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to 1% by weight of the total cannabinoid content of the dry weight of plant material; and/or
(iv) a level of total CBC of from about 0.01% to about 10%, preferably from about 0.02% to about 10%, preferably from about 0.03% to about 10%, preferably from about 0.04% to about 10%, preferably from about 0.05% to about 10%, preferably from about 0.06% to about 10%, preferably from about 0.07% to about 10%, preferably from about 0.08% to about 10%, preferably from about 0.09% to about 10%, or more preferably from about 0.1% to about 10% by weight of the total cannabinoid content of the dry weight of plant material; and/or
(v) a level of total CBN of from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material; and/or
(vi) a level of total THCV of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

### Cluster 4 Cannabinoid Profile

The term “cluster 4 cannabinoid profile” as used herein refers to a cannabinoid profile that is characterised by a level of total THC, CBG and THCV in the plant material that is greater than the level of other minor cannabinoids, such as total CBD. Accordingly, the cannabis plant of the invention may be variously described as “high-THC/CBG/THCV”, “THC-, CBG- and THCV-enriched” or “high-THC, -CBG and -THCV, low-CBD”. Those skilled in the art would understand this terminology to mean a cannabis plant that produced higher levels of THC and/or THCA, CBG and/or CBGA and THCV and/or THCVA, relative to the level of other minor cannabinoids, such as CBD.

“Tetrahydrocannabivarinic acid” or “THCVA” is derived from cannabigerovarinic acid (CBGVA), which is processed to THCVA by THCV synthase. Its neutral form, “tetrahydrocannabivarin” or “THCV” is a homologue of THC, with the substitution of a propyl side chain instead of the pentyl group on THC. As a result, the effects of THCV are distinct from THC. At low concentrations, THCV is predicted to act as an antagonist of CB1, however, at high concentrations, THCV can switch to behaving as a CB1 agonist, which is similar to the activity of THC.

In an embodiment, the level of total THC is at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material.

In an embodiment, the level of total CBG is from about 1% to about 10%, preferably from about 1% to about 9%, or more preferably from about 1% to about 8% by weight of the total cannabinoid content of the dry weight of plant material.

In an embodiment, total THC and total CBG are present at a ratio of from about 10:1 to about 100:1, preferably from about 10:1 to about 90:1, or more preferably from about 10:1 to about 80:1 (THC:CBG).

In an embodiment, the level of total THCV is from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material.

In an embodiment, total THC and total THCV are present at a ratio of from about 10:1 to about 100:1, preferably from about 10:1 to about 90:1, preferably from about 10:1 to about 80:1, preferably from about 10:1 to about 70:1, or more preferably from about 10:1 to about 50:1 (THC:THCV).

In an embodiment, the reference cannabinoid is total CBD. In another embodiment, total THC is present at a ratio of from about from about 100:1 to about 400:1 to the level of total CBD, preferably from about 110:1 to about 400:1, preferably from about 120:1 to about 400:1, preferably from about 100:1 to about 390:1, preferably from about 100:1 to about 380:1, preferably from about 100:1 to about 370:1, preferably from about 100:1 to about 360:1, preferably from about 100:1 to about 350:1, or more preferably from about 120:1 to about 350:1 (THC:CBD).

In another embodiment, the level of total CBD is from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the reference cannabinoid is total CBC. In another embodiment, the level of total THC is present at a ratio of from about from about 10:1 to about 100:1 to the level of total CBC, preferably from about 11:1 to about 100:1, preferably from about 12:1 to about 100:1, preferably from about 13:1 to about 100:1, preferably from about 14:1 to about 100:1, or more preferably from about 15:1 to about 100:1 (THC:CBC).

In another embodiment, the level of total CBC is from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material.

In an embodiment, the reference cannabinoid is total CBN. In another embodiment, the level of total THC is present at a ratio of from about from about 200:1 to about 1000:1 of the level of total CBN, preferably from about 200:1 to about 950:1, preferably from about 200:1 to about 900:1, preferably from about 200:1 to about 850:1, preferably from about 210:1 to about 1000:1, preferably from about 220:1 to about 1000:1, preferably from about 230:1 to about 1000:1, preferably from about 240:1 to about 1000:1, preferably from about 250:1 to about 1000:1, or more preferably from about 250:1 to about 850:1 (THC:CBN).

In another embodiment, the level of total CBN is from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the reference cannabinoid is total CBDV. In another embodiment, the level of total THC is present at a ratio of is from about 3000:1 to about 10000:1 to the level of total CBDV, preferably from about 3000:1 to about 9500:1, preferably from about 3000:1 to about 9000:1, preferably from about 3000:1 to about 8500:1, preferably from about 3000:1 to about 8000:1, preferably from about 3000:1 to about 7500:1, or more preferably from about 3000:1 to about 7000:1 (CBD:CBDV).

In another embodiment, the level of total CBDV is from about 0.01% to about 0.1%, preferably from about 0.01% to about 0.09%, preferably from about 0.01% to about 0.08%, preferably from about 0.1% to about 0.07%, preferably from about 0.1% to about 0.06%, or more preferably from about 0.1% to about 0.05% by weight of the total cannabinoid content of the of dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material;
(iv) optionally a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material;
(v) optionally a level of total CBC of from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material:
(vi) optionally a level of total CBN of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material; and
(vii) optionally a level of total CBDV of from about 0.01% to about 0.1%, preferably from about 0.01% to about 0.09%, preferably from about 0.01% to about 0.08%, preferably from about 0.1% to about 0.07%, preferably from about 0.1% to about 0.06%, or more preferably from about 0.1% to about 0.05% by weight of the total cannabinoid content of the of dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material; and
(iv) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material;
(iv) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material; and
(v) a level of total CBC of from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% A, to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material;
(iv) the level of total CBD is from about 0.01% to about 1%, preferably from about 0.02% to about 1%, preferably from about 0.03% to about 1%, preferably from about 0.04%, to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material;
(v) a level of total CBC of from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material; and
(vi) a level of total CBN of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material;
(iv) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material;
(v) a level of total CBC of from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material;
(vi) a level of total CBN of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material; and
(vii) a level of total CBDV of from about 0.01% to about 0.1%, preferably from about 0.01% to about 0.09%, preferably from about 0.01% to about 0.08%, preferably from about 0.1% to about 0.07%, preferably from about 0.1% to about 0.06%, or more preferably from about 0.1% to about 0.05% by weight of the total cannabinoid content of the of dry weight of plant material.

In an embodiment, the cannabis plant comprises:
(i) a level of total THC of at least about 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, or more preferably at least 95% by weight of the total cannabinoid content of the dry weight of plant material;
(ii) a level of total CBG of from about 1% to about 10%, preferably from about 1% to about 9%, or more preferably from about 1% to about 8% by weight of the total cannabinoid content of the dry weight of plant material;
(iii) a level of total THCV of from about 1% to about 10%, preferably from about 1% to about 9%, preferably from about 1% to about 8%, preferably from about 1% to about 7%, preferably from about 1% to about 6%, preferably from about 2% to about 10%, preferably from about 2% to about 9%, preferably from about 2% to about 8%, preferably from about 2% to about 7%, or more preferably from about 2% to about 6% by weight of the total cannabinoid content of the dry weight of plant material; and/or
(iv) a level of total CBD of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material; and/or
(v) a level of total CBC of from about 0.1% to about 10%, preferably from about 0.2% to about 10%, preferably from about 0.3% to about 10%, preferably from about 0.4% to about 10%, preferably from about 0.5% to about 10%, preferably from about 0.6% to about 10%, preferably from about 0.7% to about 10%, preferably from about 0.8% to about 10%, preferably from about 0.9% to about 10%, or more preferably from about 1% to about 10% by weight of the total cannabinoid content of the dry weight of plant material; and/or
(vi) a level of total CBN of from about 0.01% to about 1%, preferably from about 0.02% to about 1%, preferably from about 0.03% to about 1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material; and/or
(vii) a level of total CBDV of from about 0.01% to about 0.1%, preferably from about 0.01% to about 0.09%, preferably from about 0.01% to about 0.08%, preferably from about 0.1% to about 0.07%, preferably from about 0.1% to about 0.06%, or more preferably from about 0.1% to about 0.05% by weight of the total cannabinoid content of the of dry weight of plant material.

### Terpenes

The term “terpene” as used herein, refers to a class of organic hydrocarbon compounds, which are produced by a variety of plants. Cannabis plants produce and accumulate different terpenes, such as monoterpenes and sesquiterpenes, in the glandular trichomes of the female inflorescence. The term “terpene” includes “terpenoids” or “isoprenoids”, which are modified terpenes that contain additional functional groups.

Terpenes are responsible for much of the scent of cannabis flowers and contribute to the unique flavour qualities of cannabis products. Terpenes will be known to persons skilled in the art, illustrative examples of which are provided in Table 2.

Terpene biosynthesis in plants typically involves two pathways to produce the general 5-carbon isoprenoid diphosphate precursors of all terpenes: the plastidial methylerythritol phosphate (MEP) pathway and the cytosolic mevalonate (MEV) pathway. These pathways control the different substrate pools available for terpene synthases (TPS).

### Terpene Profile

The term “terpene profile” as used herein refers to a representation of the type, amount, level, ratio and/or proportion of terpenes that are present in the cannabis plant or part thereof, as typically measured within plant material derived from the plant or plant part, including an extract therefrom.

The terpene profile in a cannabis plant will be determined based on genetic, environmental and developmental factors, therefore particular terpenes may be present at various amounts, levels, ratios and/or proportions at any given time (i.e., at propagation, growth, harvest, drying, curing, etc).

In an embodiment, the terpene profile comprises monoterpenes and sesquiterpenes. In another embodiment, the terpene profile comprises monoterpenes. In another embodiment, the terpene profile comprises sesquiterpenes.

In an aspect disclosed herein, there is provided a method for determining a monoterpene profile of cannabis plant material, the method comprising:
a. obtaining a reference spectrum of known volatile monoterpene components;
b. selectively extracting one or more monoterpenes from the cannabis plant material;
c. obtaining spectroscopic data from the monoterpenes separated in step (b);
d. comparing the spectroscopic data to the reference spectrum to identify and/or measure the level of one or more monoterpenes to determine the monoterpene profile of the cannabis plant material.

Monoterpenes consist of two isoprene units and may be liner or contain ring structures. The primary function of monoterpenes is to protect plants from infection by fungal and bacterial pathogens and insect pests. Monoterpenes would be known to persons skilled in the art, illustrative embodiments of which include α-phellandrene, α-pinene, camphene, β-pinene, myrcene, limonene, eucalyptol, γ-terpinene and linalool.

In an embodiment, the one or more monoterpenes are selected from the group consisting of α-phellandrene, α-pinene, camphene, β-pinene, myrcene, limonene, eucalyptol, γ-terpinene and linalool. In another embodiment, the one or more monoterpenes are selected from the group consisting of α-pinene, β-pinene, myrcene, limonene and linalool. In a preferred embodiment, the one or more monoterpenes are selected from the group consisting of β-pinene and myrcene.

“Myrcene” is a monoterpinoid derivative of β-pinene. Myrcene has been associated with the therapeutic or medicinal effects of cannabis and has been suggested for use as a sedative, hypnotic, analgesic and muscle relaxant. Myrcene is also hypothesised to attenuate the activity of other cannabinoids and terpenes as part of the “entourage effect” as described in, for example, Russo, 2011. British Journal of Pharmacology, 163(7): 1344-1364.

“β-pinene” is a monoterpene that is characterised by a woody-green, pine-like smell. β-pinene has been shown to act as a topical antiseptic and a bronchodilator. β-pinene is also capable of crossing the blood-brain barrier and it is hypothesised that β-pinene inhibits the influence of THC as part of the entourage effect, as described elsewhere herein.

In an embodiment, the monoterpene profile comprises a level of monoterpenes that correlates with the level of total THC. In a preferred embodiment, the terpene profile comprises a high level of monoterpenes that correlates to high levels of THC.

In another aspect disclosed herein, there is provided a method for detecting a sesquiterpene profile of cannabis plant material, the method comprising:
a. obtaining a reference spectrum of known sesquiterpenes:
b. selectively extracting one or more sesquiterpenes from the cannabis plant material:
c. obtaining spectroscopic data from the sesquiterpenes separated in step (b);
d. comparing the spectroscopic data to the reference spectrum to identify and/or measure the level of one or more sesquiterpenes to determine the sesquiterpene profile of the cannabis plant material.

Sesquiterpenes differ from other common terpenes as they contain one additional isoprene unit, which creates a 15 carbon structure. The primary function of sesquiterpenes is as a pheromone for the bud and flower. Sesquiterpenes would be known to persons skilled in the art, illustrative embodiments of which include γ-elemene, humulene, nerolidol, guaia-3,9-diene and caryophyllene

In an embodiment, the one or more sesquiterpenes is selected from the group consisting of γ-elemene, humulene, nerolidol, guaia-3,9-diene and caryophyllene. In a preferred embodiment, the sesquiterpene is caryophyllene.

In an embodiment, the sesquiterpene terpene profile that correlates with the level of total CBD. In a preferred embodiment, the terpene profile comprises a high level of sesquiterpenes that correlates with a high level of total CBD.

### Extraction of Volatiles

It is known in the art that certain terpenes in cannabis, particularly sesquiterpenes are difficult to identify due to poor resolution a lack of reference materials (Hazekamp et al., 2016, Cannabis Cannabinoid Research, 1: 202-215). The present disclosure is predicated on the unexpected finding monoterpenes and sesquiterpenes may be selectively extracted and analysed to generate monoterpene and/or sesquiterpene profiles from cannabis plant material.

In an embodiment, the selective extraction of one or more monoterpenes from the cannabis plant material is performed using an extraction method selected from the group consisting of headspace extraction and liquid extraction.

In an embodiment, the selective extraction of one or more monoterpenes from the cannabis plant material is performed using an extraction method selected from the group consisting of headspace extraction and hexane-based liquid extraction.

Headspace extraction methods may be used for the qualitative or quantitative analysis of volatile species in samples that can be efficiently partitioned in the headspace gas volume from either liquid or solid matrices. Suitable headspace extraction methods would be known to persons skilled in the art, illustrative examples of which include static and dynamic headspace techniques.

In an embodiment, the headspace extraction method is selected from the group consisting of headspace solid phase micro-extraction (SPME) and static headspace extraction. In a preferred embodiment, the headspace extraction method is static extraction.

In an embodiment, the selective extraction of one or more sesquiterpenes from the cannabis plant material is performed by liquid extraction.

Liquid extraction is a common sample preparation method that is used in targeted chemotypic analysis. Suitable liquid extraction methods would be known to persons skilled in the art, illustrative examples of which include MeOH-, hexane-, DCM-based liquid extraction.

In an embodiment, the selective extraction of one or more monoterpenes from the cannabis plant material is performed by hexane-based liquid extraction.

In an embodiment, the selective extraction of one or more sesquiterpenes from the cannabis plant material is performed by hexane-based liquid extraction.

As described elsewhere herein, the use of hexane-based liquid extraction is particularly advantageous for absolute quantification of terpenes (i.e., monoterpenes and sesquiterpenes) in accordance with the methods disclosed herein.

Accordingly, in an embodiment, the liquid extraction is a hexane-based liquid extraction.

### Gas Chromatography/Mass Spectrometry (GC-MS)

The present disclosure provides methods for determining a terpene profile of cannabis plant material from spectroscopic data. Methods for measuring spectroscopic data would be known to persons skilled in the art, illustrative examples of which include gas chromatography/mass spectroscopy (MS).

In an embodiment, the spectroscopic data are measured using GC-MS.

The term “spectroscopic data” as used herein refers to a MS spectrum or spectra. MS spectra can be used to identify single chemical characteristics of a certain chemical group (i.e., terpenes) and more complex characteristics, such as the chemical, structural, sensoric or functional qualities of different cannabis plants.

Apparatus for measuring spectroscopic data would be known to persons skilled in the art, illustrative examples of which include a GC-QQQ system as described elsewhere herein. GC-MS apparatus comprise a gas chromatograph and mass spectrometer. The gas chromatograph comprises a capillary column.

In an embodiment, the gas chromatography is performed using a DB-5 capillary GC column.

Persons skilled in the art would recognise that isolated cannabinoids and terpenes may exists as a number of different chemical species, illustrative examples of which include salts, solvates, prodrugs, stereoisomers or tautomers thereof.

### Methods for Selecting Cannabis Plants

In another aspect, there is provided a method for selecting a cannabis plant comprising a terpene profile enriched for monoterpenes, the method comprising:
a. harvesting plant material from a plurality of different cannabis plants;
b. at least partially drying the harvested plant material of step (a);
c. measuring in the at least partially dried plant material of step (b) a level of one or more monoterpenes and to generate a monoterpene profile for each of the plurality of cannabis plants in accordance with the method as described herein; and
d. on the basis of the measurements from step (c), selecting from the plurality of different cannabis plants a cannabis plant comprising a terpene profile that is enriched for monoterpenes.

The terms “selecting” or “selection” as used herein means the selection of one or more cannabis plants from the plurality of different cannabis plants based on the terpene profile of the individual cannabis plant. The term “plurality” is to be understood to mean more than 1 (e.g., 2 3, 4, 5, 6, 7, 8, 9, 10, 11, etc.).

In an embodiment, the method further comprises the selection of one or more cannabis plants based on the cannabinoid profile of the individual cannabis plants.

In an embodiment, the selected cannabis plant comprising a terpene profile enriched for monoterpenes further comprises a predetermined cannabinoid profile that is selected from the group consisting of (i) a cluster 2 cannabinoid profile: (ii) a cluster 3 cannabinoid profile; and (iii) a cluster 4 cannabinoid profile.

In another aspect, there is provided a method selecting a cannabis plant comprising a terpene profile enriched for sesquiterpenes, the method comprising:
a. harvesting plant material from a plurality of different cannabis plants;
b. at least partially drying the harvested plant material of step (a);
c. measuring in the at least partially dried plant material of step (b) a level of one or more sesquiterpenes and to generate a sesquiterpene profile for each of the plurality of cannabis plants in accordance with the method as described herein; and
d. on the basis of the measurements from step (c), selecting from the plurality of different cannabis plants a cannabis plant comprising a terpene profile that is enriched for sesquiterpene.

In an embodiment, the selected cannabis plant comprising a terpene profile enriched for sesquiterpenes further comprises a predetermined cannabinoid profile that is selected from the group consisting of (i) a cluster 1 cannabinoid profile; and (ii) a cluster 2 cannabinoid profile.

### Methods for Monitoring a Cannabis Plant

The methods disclosed herein may suitably be used to monitor changes to the terpene profile of cannabis plants, for example, during their growth cycle. This advantageously allows breeders, cultivators and the like to monitor their crop to ensure their plants retain/maintain the desired terpene profile(s) and, where necessary, remove and/or discard plants with an undesirable terpene profile.

Thus, in another aspect disclosed herein, there is provided a method for monitoring a cannabis plant for a change in the terpene profile of the cannabis plant, the method comprising:
a. determining the terpene profile of plant material derived from a cannabis plant in accordance with the methods disclosed herein; and
b. determining the terpene profile of plant material derived from the same cannabis plant as (a) in accordance with the methods disclosed herein and at a subsequent time point in the growth cycle of the plant;
c. comparing the terpene profiles determined at (a) and (b) to evaluate whether there has been a change to the terpene profile of the cannabis plant.

### Methods of Selecting Growing Conditions

The methods disclosed herein may also suitably be used to select growing conditions (e.g., frequency of watering, water quantity and/or quality; amount and/or type of fertiliser used; etc.) that give rise to or promote the development of cannabis plants with a desired terpene profile. This advantageously allows breeders, cultivators and the like to optimise growing conditions to produce cannabis plants with desired terpene profile(s).

Thus, in another aspect disclosed herein, there is provided a method of selecting growing conditions that favour the development of a cannabis plant with a desirable chemotypic profile, the method comprising: the method comprising:
a. exposing a first cannabis plant to a first set of selected growing conditions for a period of time:
b. exposing a second cannabis plant to a second set of growing conditions for a period of time, wherein the second set of selected growing conditions is different from the first set of growing conditions;
c. optionally, repeating step (b) for a subsequent set of growing conditions that is different from the first and second sets of selected growing conditions;
d. determining the terpene profile(s) of plant material derived from the cannabis plants exposed to the set of selected growing conditions of steps (a)-(c) in accordance with the methods disclosed herein; and
e. selecting from the set of growing conditions of steps (a)-(c) one or more sets of selected growing conditions that favour the development of a cannabis plant with a desirable terpene profile based on the terpene profiles determined at step (d).

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### Examples

### A. Terpene Analysis

### Reagents and Standards

Primary standards were commercially purchased from Sigma-Aldrich (Castle Hill, Australia) except for bisabolol and fenchone which were obtained via Novachem Pty Ltd (Heidelberg West, Australia) as distributor for AccuStandard (Connecticut, USA). The standards tested are detailed in Table 2. All solvents, hexane, methanol and dichloromethane were obtained from Fisher Scientific (Fair Lawn, N.J.).

TABLE 2
Terpenes standards used to identify terpenes.

| Peak | RT | | | Retention |
|---|---|---|---|---|
| No. | (min) | Name | m/z | Index |
| 1 | 8.849 | α-Phellandrene | 93.0 | 928 |
| 2 | 9.092 | α-Pinene (+/−) | 93.0 | 937 |
| 3 | 9.590 | Camphene | 93.0 | 955 |
| 4 | 10.383 | β-Pinene (+/−) | 93.0 | 983 |
| 5 | 10.570 | Myrcene | 93.0 | 990 |
| 6 | 11.481 | α-Terpinene | 93.0 | 1021 |
| 7 | 11.848 | Limonene | 68.1 | 1033 |
| 8 | 11.930 | β-Phellandrene | 93.1 | 1036 |
| 9 | 11.983 | Eucalyptol | 81.0 | 1038 |
| 10 | 12.264 | Ocimene isomer | 93.1 | 1047 |
| 11 | 12.700 | γ-Terpinene | 93.1 | 1061 |
| 12 | 13.088 | 4-Thujanol | 93.1 | 1074 |
| 13 | 13.531 | Terpinolene | 93.1 | 1089 |
| 14 | 13.708 | Fenchone | 81.1 | 1095 |
| 15 | 13.868 | Linalool | 93.0 | 1101 |
| 16 | 14.615 | Fenchol | 81.1 | 1126 |
| 17 | 14.844 | Trans-2-Pinanol | 93.1 | 1133 |
| 18 | 16.219 | Borneol | 95.1 | 1180 |
| 19 | 16.835 | α-Terpineol | 93.1 | 1200 |
| 20 | 22.511 | α-Bergamotene | 93.1 | 1406 |
| 21 | 22.854 | β-Bergamotene | 119.1 | 1419 |
| 22 | 23.148 | trans- | 93.0 | 1431 |
| | | Caryophyllene | | |
| 23 | 23.280 | γ-Elemene iso1 | 121.0 | 1436 |
| 24 | 23.360 | Bergamontene | 93.1 | 1439 |
| | | iso3 | | |
| 25 | 23.467 | α-Guaiene | 105.0 | 1443 |
| 26 | 23.748 | Farnesene | 69.2 | 1454 |
| 27 | 24.083 | Humulene | 93.0 | 1467 |
| 28 | 24.780 | (−)-α-Selinene | 105.1 | 1494 |
| 29 | 24.932 | epi-β-selinene | 93.1 | 1500 |
| 30 | 25.050 | sesquiT- | 93.1 | 1510 |
| | | coeluting01 | | |
| 31 | 25.174 | δ-Guaiene | 107.0 | 1520 |
| 32 | 25.999 | α-Bisabolene | 93.1 | 1545 |
| 33 | 26.099 | Guaia-3,9-diene | 161.1 | 1549 |
| 34 | 26.210 | 3,7(11)- | 161.1 | 1553 |
| | | Selinadiene | | |
| 35 | 26.456 | β-cis- | 69.2 | 1563 |
| | | Caryophyllene | | |
| 36 | 26.660 | γ-Elemene iso2 | 121.1 | 1572 |
| 37 | 27.242 | Caryophyllene | 79.0 | 1596 |
| | | oxide | | |
| 38 | 27.474 | Guaiol | 105.1 | 1606 |
| 39 | 28.187 | β-Cadinene | 189.1 | 1637 |
| 40 | 28.922 | γ-Guriunene | 59.1 | 1669 |
| 41 | 29.081 | sesquiterpene | 107.0 | 1676 |
| 42 | 29.455 | α-Bisabolol | 93.0 | 1692 |

### Sample Preparation

Dried and ground plant material was obtained from the Victorian Government Medicinal Cannabis Cultivation Facility. Mature buds (aged from three to five weeks depending on the strain) from 69 different cannabis cultivars were analysed. Samples were ground to a fine powder with liquid nitrogen using a SPEX SamplePrep 2010 Geno/Grinder for 1 minute at 1500 rpm. For static head space analysis 20 mg of each sample was weighed into a 15 mL head space vial. For liquid extractions, 20 mg of sample was weighed into an Axygen 2.0 mL microcentrifuge tube on a Sartorius BP210D analytical balance. For method optimisation a composite sample (five diverse cannabis strains) was extracted with methanol, hexane or dichloromethane (2×200 μL), sonicated for 5 minutes and centrifuged at 13,000 rpm for 5 minutes. The supernatants were combined and transferred to a 2 mL amber HPLC vial.

### Gas Chromatography/Mass Spectroscopy (GC-MS) Analysis

Samples were analysed using an Agilent 7000 GC-QQQ system equipped with EPC backflush option and a Gerstal MultiPurpose Sampler MPS.

Separation trialed using a range of columns including was carried out using 30 m capillary GC columns with different stationary phases: DB-5, DB-17 or VF-35. Columns were obtained from Trenbio (Alphington, Australia) as distributor for Agilent (Santa Clara, USA). The separation gradients are described in Table 3 and 4. The MS was set to acquire a full range spectrum (29-350 m/z).

TABLE 3
HS- and GC-MC parameters used for relative
determination of terpenes from cannabis.
GC-MS parameters for static headspace analysis

| | |
|---|---|
| Sample | 20 mg milled, dried cannabis bud |
| Incubation (pre- | 5 min |
| extraction) | |
| Extraction | 30 min |
| Desorption | 2 min |
| Fibre post-bake | 30 min |

GC-MS Parameters

| Column | DB-5, DB-17, VF-35 | |
|---|---|---|
| Oven | Time (min) | Temperature (° C.) |
| | 0 | 60 |
| | 2 | 60 |
| | 7 | 110 |
| | 12 | 110 |
| | 13.5 | 125 |
| | 18.5 | 125 |
| | 26 | 200 |
| | 28 | 300 |
| | 31 | 300 |
| | TOTAL RUN TIME | 31 min |

| | |
|---|---|
| Carrier gas | Helium, Flow: 1.6221 mL/min at 1.5 psi |
| Injector | 10:1 split, 250° C., injection volume 1000 μL |
| Headspace | Incubation temperature 130° C. for 5 min, |
| | injection volume 1000 μL |
| Scan range | 29-350 m/z |

TABLE 4
Liquid extraction and GC-MS parameters used for quantitative
determination of terpenes from cannabis.
GC-MS parameters for liquid injection analysis

| Sample | 20 mg milled, dried cannabis bud, extracted twice | |
|---|---|---|
| | with 200 μL hexane. Extracts combined for analysis | |
| Oven | Time (min) | Temperature (° C.) |
| | 0 | 60 |
| | 2 | 60 |
| | 30 | 200 |
| | 31 | 320 |
| | 34 | 320 |
| | TOTAL RUN TIME | 35 min |

| | |
|---|---|
| Carrier gas | Helium, Flow: 1.6221 mL/min at 1.5 psi |
| Injector | 1:5 split, 250° C., 1 μL |
| Flow rates and | Main Run |
| Backflush | Column 1: flow 1.3801 mL/min; average velocity |
| settings | 32.251 cm/sec; |
| | Column 2: 0.50013 psi, flow 1.428 mL/min; average |
| | velocity 151.6 cm/sec |
| | Backflush |
| | Column 1: 3 min (post-run) - 1.9815 mL/min; |
| | Column 2: 3 min (post-run) - 7.0194 mL/min. |

### Data Processing

### Relative Quantitation

Chromatograms were processed using Agilent MassHunter software by comparison with the retention time and mass spectrum of the standards (Table 2).

### Chemometric Analysis

GCMS data was analysed in PLSToolbox (Version 8.6.1, Eigenvector Research, Inc.) running on MatLab (Version R2018a, Mathworks).

### Results

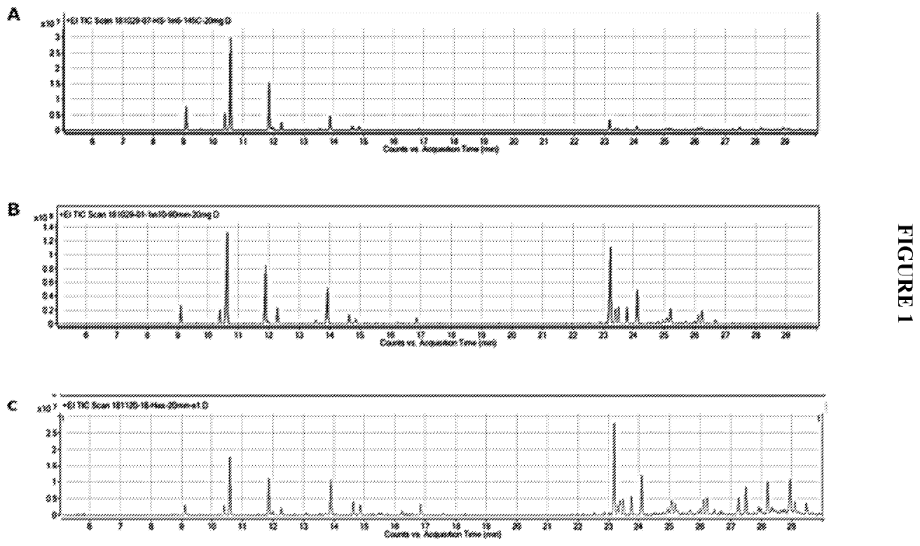

Terpenes were extracted from 20 mg of milled, dried cannabis biomass using static headspace with direct injection, headspace solid phase micro-extraction (SPME) or liquid extraction using hexane followed by chromatographic separation on an Agilent 7000 GC-QQQ using a DB-5, DB-17 or VF-35 capillary GC column. The optimal column for separation of the volatiles was the DB-5 column. Static headspace was effective for the analysis of monoterpenes, while sesquiterpenes were more effectively extracted using a hexane-based liquid approach (FIG. 1).

Peak identifications were assigned using MS spectral matching against reference spectra in the NIST/Wiley libraries and Kovats Indicies. Confirmatory identification was done based on retention index, which was calculated for the compounds identified in each sample using an external standard analysed under the same GC conditions. The external standards (Table 2) enabled the assignment of major volatile peaks in the cannabis strains (Table 4). Several peaks were not able to be identified with certainty by library matching or by comparison to the standards. These include both putative monoterpenes (M01-M13) and sesquiterpenes (S01-S08).

TABLE 5
Terpenes identified in the current cannabis collection and their properties.

| Terpene | | | Mass/Charge |
|---|---|---|---|
| No. | Name | Structure | number (m/z)* |
| 1 | α-Phellandrene | 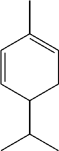 | m/z 93.0 |
| 2 | α-Pinene (+/−) | 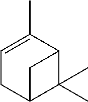 | m/z 93.0 |
| 3 | Camphene | 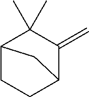 | m/z 93.0 |
| 4 | β-Pinene (+/−) | 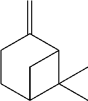 | m/z 93.0 |
| 5 | Myrcene | 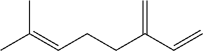 | m/z 93.0 |
| 6 | Limonene | 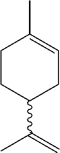 | m/z 68.1 |
| 7 | Eucalyptol | 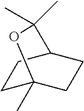 | m/z 81.0 |
| 8 | γ-Terpinene | 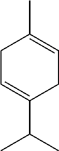 | m/z 93.1 |
| 9 | Linalool | 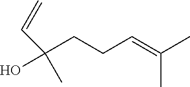 | m/z 93.0 |
| 10 | γ-Elemene | 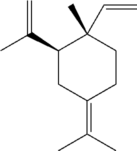 | m/z 121.0 |
| 11 | Humulene | 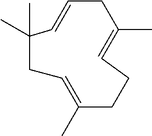 | m/z 93.0 |
| 12 | Nerolidol | 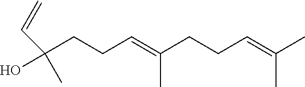 [SMILES: C=CC(C)(O)CC/C=C(\C)CCC=C(C)C] | m/z 222.4 |
| 13 | Guaia-3,9-diene | 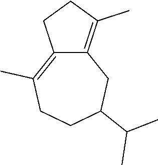 | m/z 161.1 |
| 14 | Caryophyllene | 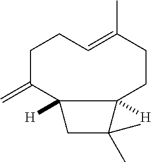 | m/z 69.2 |

*The molecular ion is not necessarily seen for all compounds.

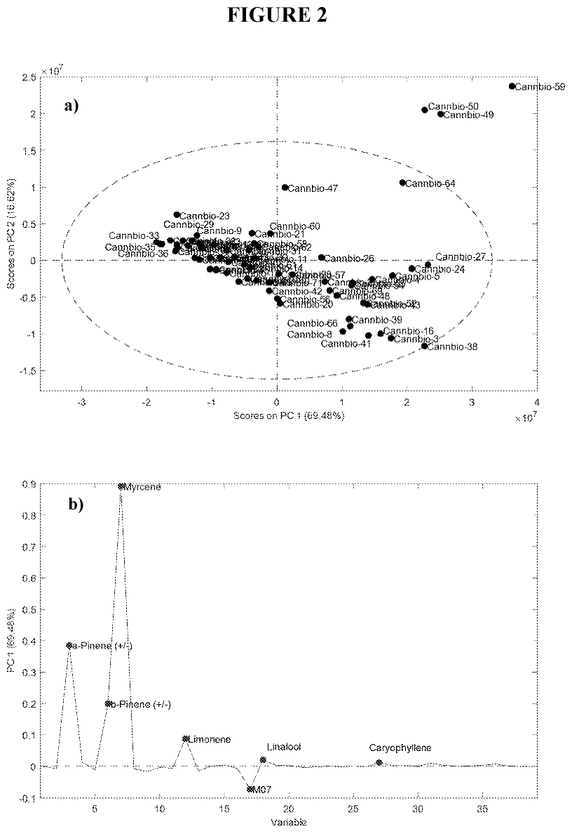

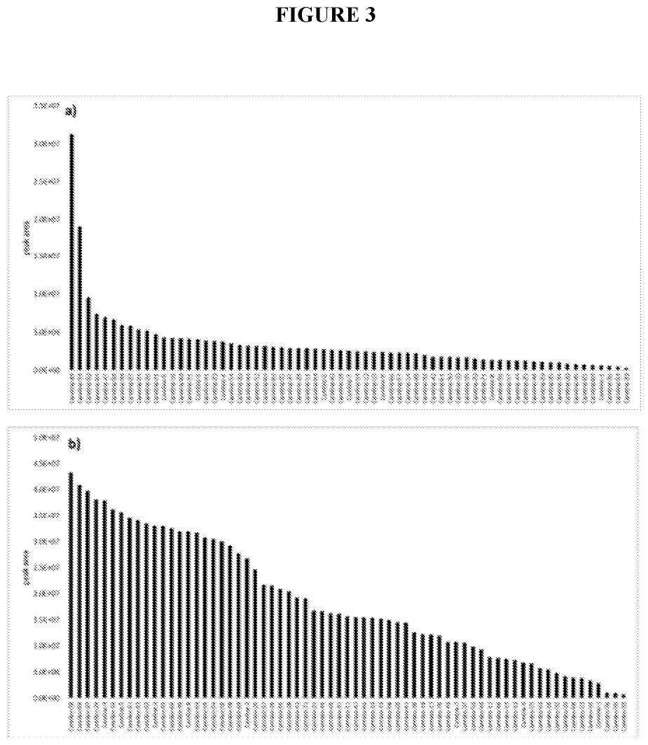

Principal components analysis (PCA) suggests there is considerable diversity within in strain collection (FIG. 2). In this static headspace analysis the most influential terpenes contribution to the PCA separation are myrcene, limonene, α- and β-pinene. The abundance of these terpenes vary within the population diversity as exemplified by the data for myrcene and β-pinene (FIG. 3).

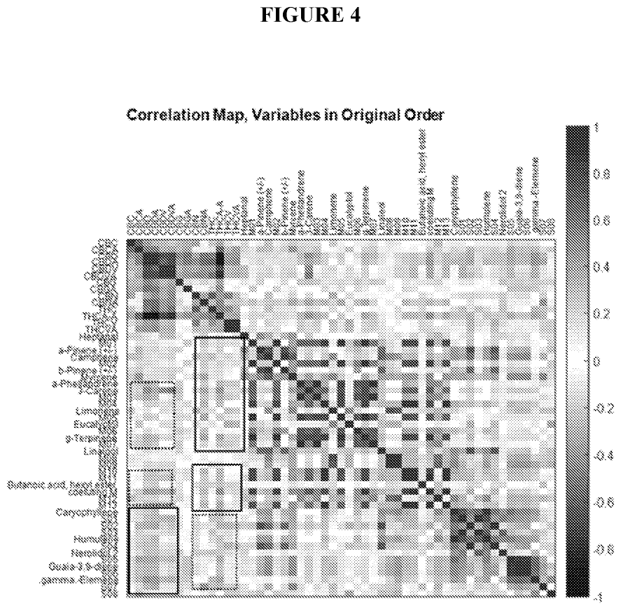

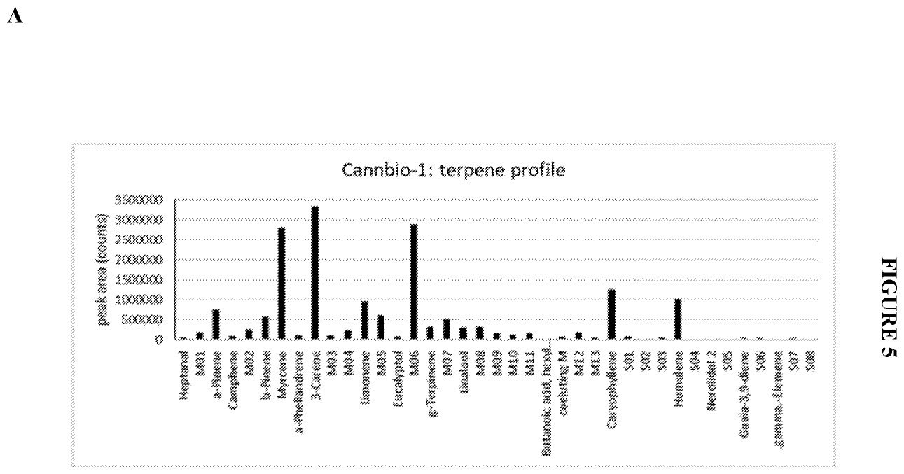

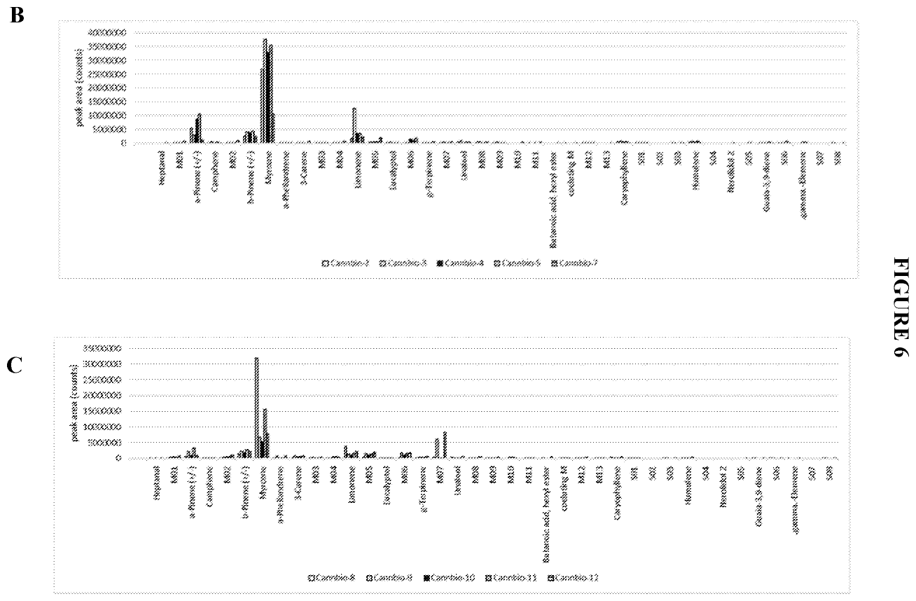

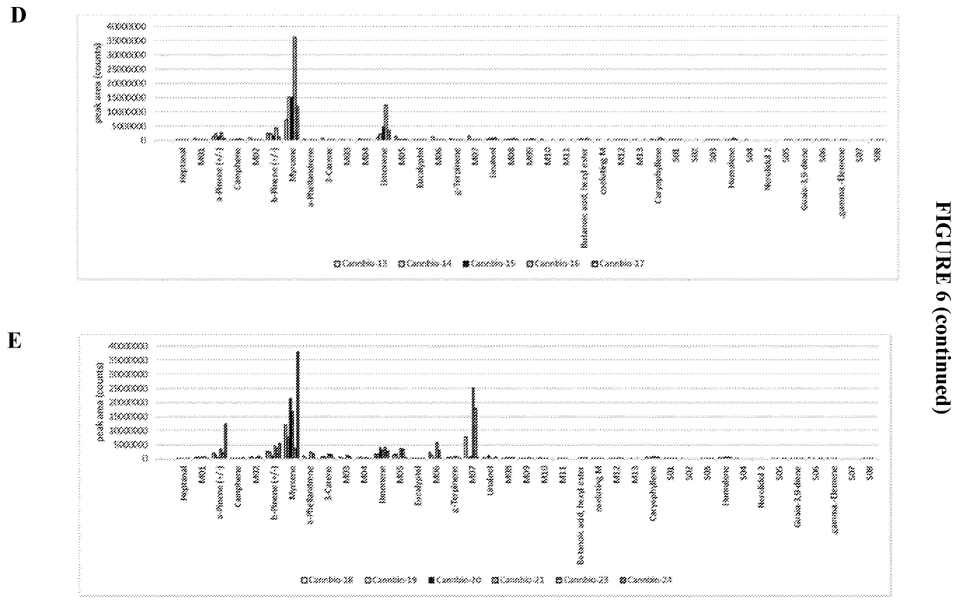

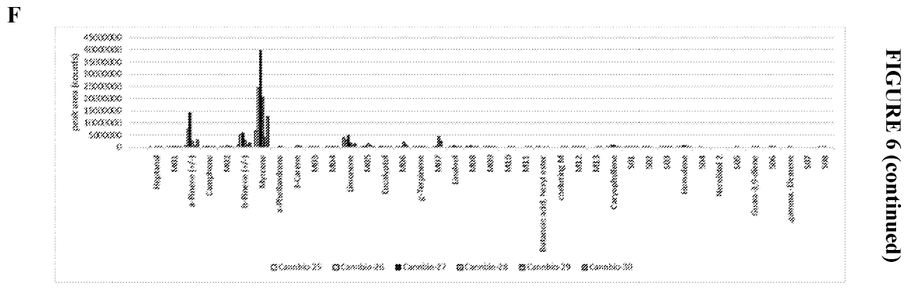

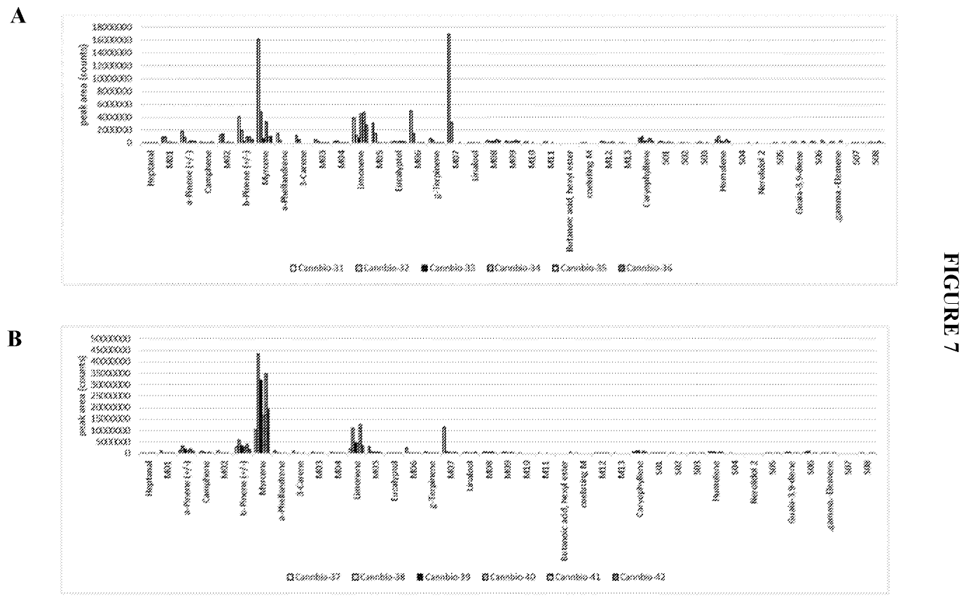

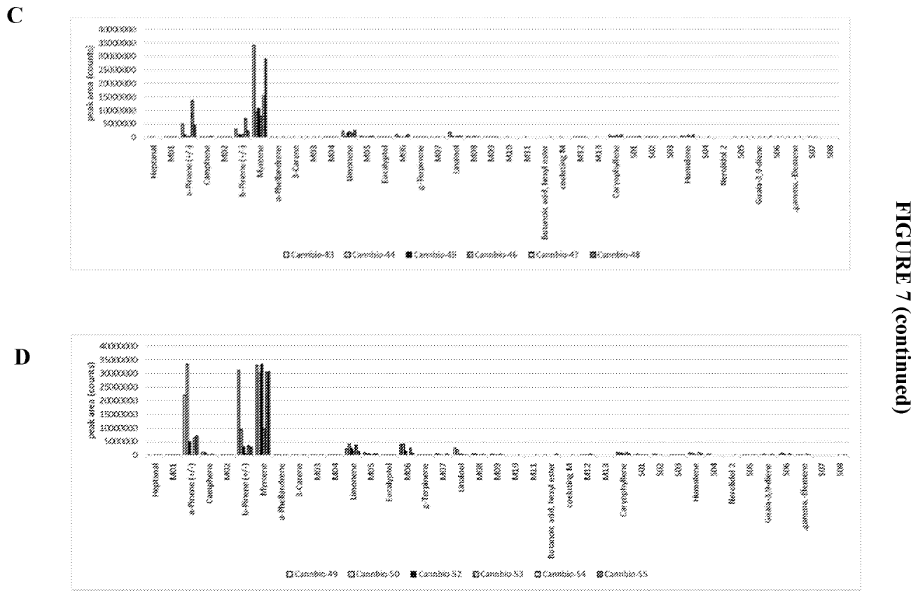

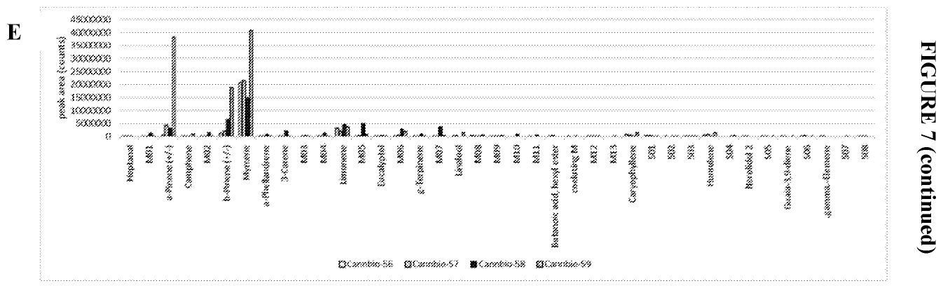

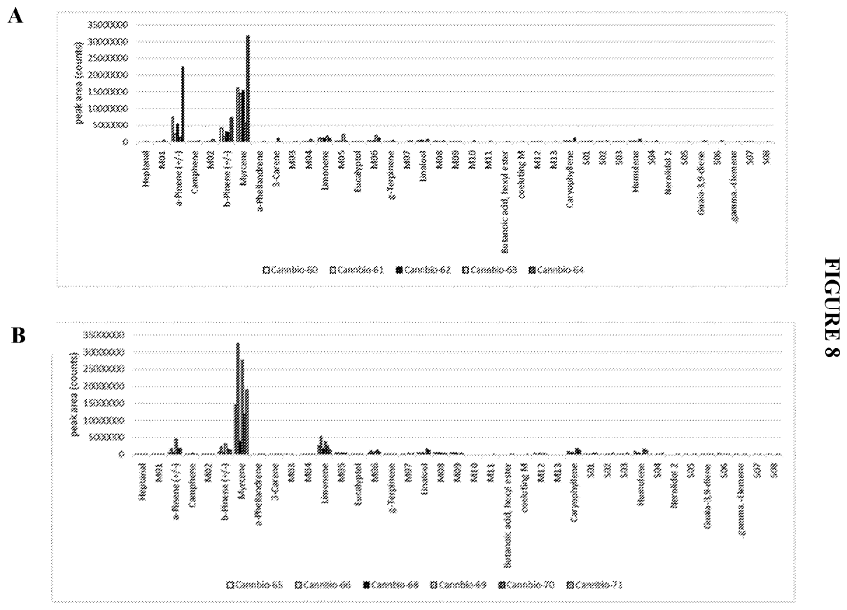

Despite the population diversity, we surprisingly identified correlations between different groups of terpenes and cannabinoids (FIG. 4). The strains that tend to be high in THC have more of the monoterpenes compared the sesquiterpenes whereas high CBD tends to correlate to higher sesquiterpenes.

### B. Cannabinoid Analysis

### Plants

Cannabis plants were grown under an Office of Drug Control license at the Victorian Government Medicinal Cannabis Cultivation Facility, Victoria, Australia. Indoor greenhouse growing facilities were equipped with full climate control (i.e., temperature, humidity and high-intensity lighting) to ensure that crops were produced in almost identical growing conditions.

Cannabis plants were asexually propagated from cuttings taken from vegetative mother plants originating from a single seed source. Cuttings were maintained for 2 weeks at 22° C. in a high humidity environment (i.e., 50% relative humidity) under 18 hours day light in rooting medium to stimulate root development before being transferred to substrate medium for hydroponic growth. The plants were grown for a further 5 weeks under the same growth conditions before being transferred to a larger substrate medium to induce flowering.

Flowering conditions were identical to the rooting and growth conditions, with the exception that the day light length was reduced to 12 hours. The plants were maintained in flowing conditions for 9 weeks to allow for flowering and maturation.

The plants were irrigated throughout their growing cycle with potable quality water and sustained release fertilizer is applied to the soil-free medium.

Upon maturation, plants were harvested at the base of the plant and dried in a temperature and humidity controlled environment (i.e., approximately 21° C. at approximately 38-45% humidity) for between 3 to 5 weeks prior to extraction or analysis, as described below.

### Reagents and Standards

All HPLC grade reagents, water with 0.1% formic acid (mobile phase A), acetonitrile with 0.1% formic acid (mobile phase B) and methanol were obtained from Fisher Scientific (Fair Lawn, N.J.). Primary standards for CBDA and THCA in acetonitrile, and CBD, CBN, CBC, THC in methanol, at 1000 μg/mL, were commercially purchased from Novachem Pty Ltd (Heidelberg West, Australia) as distributor for Cerilliant Corporation (Round Rock, Tex.). A mixed stock standard at 125 μg/mL CBDA, CBN, CBC. THCA and 250 μg/mL CBD, THC in methanol was prepared with working standards at 0.05, 0.125, 0.25, 0.5, 1.25, 2.5 and 50.0 μg/mL for CBDA, CBN, CBC and THCA; and 0.1, 0.25, 0.5, 1.0, 2.5, 5.0 and 100.0 μg/mL for CBD and THC prepared from the mixed stock. Primary standards for THCV, CBDV, CBG, THCVA, CBNA, CBCA, CBGA, CBL and A8-THC in methanol, at 1000 μg/mL, were commercially purchased from Novachem Pty Ltd (Heidelberg West, Australia) as distributor for Cerilliant Corporation (Round Rock, Tex.). These were combined to make a 100 μg/mL stock (i.e. 100 uL taken and mixed from each). This mixed standard was diluted to 0.1, 0.25, 0.5, 1.0, 2.5, 5.0 and 100 μg/mL. All standards were stored at −80° C.

### Sample Preparation

Inflorescences were separated from the plant material from 69 different female cannabis cultivars. Samples were ground to a fine powder with liquid nitrogen using a SPEX SamplePrep 2010 Geno/Grinder for 1 minute at 1500 rpm. After grinding, 10 mg of each sample was weighed into an Axygen 2.0 mL microcentrifuge tube on a Sartorius BP210D analytical balance. Each sample was extracted with 1 mL of methanol, vortexed for 30 seconds, sonicated for 5 minutes and centrifuged at 13,000 rpm for 5 minutes. The supernatant was transferred to a 2 mL amber HPLC vial and diluted 1:3 for analysis.

Where necessary, plant material was cured by heating the ground dried plant material at 120° C. for 2 hours.

### Liquid Chromatography/Mass Spectroscopy Analysis

Samples were analysed using a Thermo Scientific (Waltham, Mass.) Q Exactive Plus Orbitrap mass spectrometer (MS) coupled with Thermo Scientific Vanquish ultra-high performance liquid chromatography (UHPLC) system equipped with degasser, binary pump, temperature controlled autosampler and column compartment, and photodiode array detector (PDA).

Separation was carried out using a C18 column (Phenomenex Luna Omega, 1.6 μm, 150 mm×2.1 mm) maintained at 30° C. with water and acetonitrile (both with 0.1% formic acid) as mobile phases and a flow rate of 0.3 mL/min. The separation gradient is described in Table 6.

The MS was set to acquire a full range spectrum (80-1,200 m/z) followed by a data independent MS2 spectrum in positive polarity with resolution set to 35,000. The capillary temperature was set to 320° C. with sheath and auxiliary gas at 28 and 15 units respectively and a spray voltage of 4 kV. PDA data acquisition was set to a data collection rate of 5 Hz from 190 and 680 nm.

TABLE 6
Separation gradient for LCMS analysis.

| Time | % A (Water | % B (Acetonitrile |
|---|---|---|
| (min) | with 0.1% FA) | with 0.1% FA) |
| 0 | 60.0 | 40.0 |
| 2.0 | 60.0 | 40.0 |
| 3.0 | 25.0 | 75.0 |
| 10.0 | 10.0 | 90.0 |
| 11.0 | 0.0 | 100.0 |
| 15.0 | 0.0 | 100.0 |
| 15.1 | 60.0 | 40.0 |
| 20.0 | 60.0 | 40.0 |

### Data Processing

### Chemometric Analysis

LCMS data was aligned, peaks picked and isotopes clustered in Genedata Refiner MS (Genedata Expressionist® 11.0.0a, Basel, Switzerland). The subsequent cluster volumes were analysed in Genedata Analyst. A total of 2,734 clusters were identified.

TABLE 7
Cannabinoid standards used for profiling.

| Cannabinoid | Formula | Charge | m/z | RT |
|---|---|---|---|---|
| Tetrahydrocannabivarin | C₁₉H₂₆O₂ | 1 | 287.2004 | 7.90 |
| (THCV) | | | | |
| Cannabidivarin (CBDV) | C₁₉H₂₆O₂ | 1 | 287.2006 | 6.45 |
| Cannabinol (CBN) | C₂₁H₂₆O₂ | 1 | 311.2005 | 8.92 |
| Cannabidiol (CBD) | C₂₁H₃₀O₂ | 1 | 315.2316 | 7.65 |
| (−)-Δ9-Tetrahydrocannabinol | C₂₁H₃₀O₂2 | 1 | 315.2317 | 9.76 |
| (THC) | | | | |
| Cannabichromene (CBC) | C₂₁H₃₂O₂ | 1 | 317.2110 | 10.89 |
| Cannabigerol (CBG) | C₂₁H₃₂O₂ | 1 | 317.2473 | 7.52 |
| Tetrahydrocannabivarinic acid | C₂₀H₂₆O₄ | 1 | 331.1902 | 8.70 |
| (THCVA) | | | | |
| Cannabidivarinic acid | C₂₀H₂₆O₄ | 1 | 331.1902 | 6.19 |
| (CBDVA) | | | | |
| Cannabinolic acid (CBNA) | C₂₂H₂₆O₄ | 1 | 355.1901 | 9.73 |
| Cannabidiolic acid (CBDA) | C₂₂H₃₀O₄ | 1 | 359.2214 | 7.16 |
| Cannabichromenic acid | C₂₂H₃₀O₄ | 1 | 359.2216 | 11.18 |
| (CBCA) | | | | |
| Δ9-Tetrahydrocannabinolic | C₂₂H₃₀O₄ | 1 | 359.4439 | 10.73 |
| acid (THCA) | | | | |
| Cannabigerolic acid (CBGA) | C₂₂H₃₂O₄ | 1 | 361.2371 | 7.41 |

### Quantitation

Chromatograms were processed using Thermo LCQuan v.2.7 software by extracted ion using the n/z values specified in Table 7 with a window of 5 ppm or by PDA analysis at 280 nm. Calibration curves were developed using the serial diluted standards and the amount of each cannabinoid in the cultivars calculated.

### Results

### Chemotyping

LCMS analysis was undertaken to identify plants with cannabinoid profiles enriched for total CBD. For untargeted analysis the intensity cut off was stringent, meaning only peaks that were relatively intense would be selected. Post peak alignment and isotope clustering a total of 2,734 isotope clusters were identified in the combined dataset. Since standards were run under the same conditions along with the plant extracts, cannabinoid profiles were generated corresponding to the known cannabinoids (Table 7). For this analysis, the plant material had not been heated so the acid forms were present at higher levels than the respective neutral species (Citti et al. 2018. Phytochemical Analysis, 29: 539-48).

### Quantitative Analysis

### Cluster 1 Cannabinoid Profile

The results obtained from this analysis indicated that the total CBD is 55.10 mg/g on a dried weight basis. Total THC is 1.89 mg/g. The other cannabinoids are also present in low levels: total CBG is 0.71 mg/g, total CBC is 2.29 mg/g, total CBN is 0.02 mg/g, total CBDV is 0.83 mg/g and total THCV is 0.10 mg/g. The results from this analysis are summarised in Table 8, below.

TABLE 8
Quantitative analysis of cannabinoids in CBD-enriched cannabis

| | Concentration | Ratio | % of total |
|---|---|---|---|
| Cannabinoid | (mg/g) | (CBD:Cannabinoid) | cannabinoid content |
| CBD | 55.1 | 1 | 90.42 |
| THC | 1.89 | 29.15 | 3.10 |
| CBG | 0.71 | 77.61 | 1.17 |
| CBC | 2.29 | 24.06 | 3.76 |
| CBN | 0.02 | 2755 | 0.03 |
| CBDV | 0.83 | 66.39 | 1.36 |
| THCV | 0.1 | 551 | 0.16 |
| TOTAL | 60.94 | | |

### Cluster 2 Cannabinoid Profile

To fully describe the cannabinoid profile enriched for total CBD and total THC, quantitative analysis was performed on the 27 cannabis strains with a cannabinoid profile enriched for total CBD and total THC. The results obtained from this analysis are provided in Table 9, below.

TABLE 9
Quantitative analysis of cannabinoids in CBD- and THC-enriched cannabis,

| Strain | | | | | | | | Total |
|---|---|---|---|---|---|---|---|---|
| # | CBD | THC | CBG | CBC | CBN | CBDV | THCV | cannabinoid |
| 2 | 53.33 | 33.96 | 1.21 | 3.12 | 0.1 | 0.23 | 0.24 | 92.19 |
| 3 | 91.42 | 57.2 | 2.52 | 5.39 | 0.05 | 0.2 | 0.32 | 157.1 |
| 6 | 55.49 | 31.36 | 2.38 | 3.08 | 0.09 | 0.3 | 0.35 | 93.05 |
| 7 | 69.26 | 36.69 | 2.36 | 4.01 | 0.11 | 0.33 | 0.29 | 113.05 |
| 8 | 74.14 | 29.76 | 3.64 | 4.39 | 0.15 | 0.37 | 0.34 | 112.79 |
| 9 | 69.51 | 33.38 | 2.55 | 3.77 | 0.13 | 0.32 | 0.35 | 110.01 |
| 10 | 51.97 | 22.68 | 2.11 | 2.71 | 0.09 | 0.29 | 0.26 | 80.11 |
| 11 | 65.71 | 35.09 | 2.24 | 3.46 | 0.09 | 0.32 | 0.29 | 107.2 |
| 12 | 70.87 | 33.14 | 3.72 | 3.99 | 0.1 | 0.37 | 0.36 | 112.55 |
| 13 | 64.27 | 30.26 | 1.9 | 3.04 | 0.13 | 0.35 | 0.32 | 100.27 |
| 14 | 78.37 | 41.58 | 4.48 | 3.94 | 0.16 | 0.41 | 0.42 | 129.36 |
| 15 | 73.06 | 38.33 | 2.37 | 3.77 | 0.07 | 0.39 | 0.45 | 118.44 |
| 16 | 96.97 | 74.48 | 5.12 | 5.22 | 0.13 | 0.47 | 0.49 | 182.88 |
| 17 | 76.72 | 36.42 | 2.86 | 4.05 | 0.1 | 0.37 | 0.31 | 120.83 |
| 18 | 67.57 | 22.29 | 2.14 | 3.7 | 0.08 | 0.41 | 0.41 | 96.6 |
| 19 | 76.61 | 37.91 | 3.64 | 4.75 | 0.1 | 0.39 | 0.35 | 123.75 |
| 20 | 86.25 | 36.4 | 3.13 | 5.07 | 0.11 | 0.43 | 0.54 | 131.93 |
| 21 | 56.72 | 20.86 | 1.06 | 3.07 | 0.08 | 0.1 | 0.27 | 82.16 |
| 22 | 68.15 | 25.38 | 1.17 | 4.12 | 0.11 | 0.12 | 0.29 | 99.34 |
| 23 | 51.19 | 20.49 | 1.9 | 3.16 | 0.09 | 0.09 | 0.16 | 77.08 |
| 24 | 74.74 | 27.35 | 1.26 | 4.24 | 0.1 | 0.14 | 0.27 | 108.1 |
| 25 | 73.92 | 38.55 | 1.95 | 4.27 | 0.12 | 0.13 | 0.25 | 119.19 |
| 26 | 82.46 | 43.34 | 1.46 | 6.21 | 0.11 | 0.2 | 0.27 | 134.05 |
| 27 | 70.43 | 50.77 | 2.77 | 4.04 | 0.08 | 0.41 | 0.33 | 128.83 |
| 28 | 65.4 | 33.14 | 0.94 | 3.41 | 0.19 | 0.31 | 0.35 | 103.74 |
| 29 | 43.1 | 22.39 | 1.17 | 2.56 | 0.11 | 0.2 | 0.21 | 69.74 |
| 30 | 42.82 | 28.36 | 1.3 | 2.22 | 0.12 | 0.23 | 0.28 | 75.33 |

Using the total cannabinoid content (mg/g) for each of the analysed cannabis strains, the proportion of each cannabinoid in the total cannabinoid content of the plant material was derived to further characterise the cannabinoid profile of the cannabis strains, presented as a percentage (%) of the total cannabinoid content of the dry weight of plant material (Table 10).

TABLE 10
Major and minor cannabinoid content in
CBD- and THC-cnriched cannabis.

| | % | % | % | % | % | % | % |
|---|---|---|---|---|---|---|---|
| Strain # | CBD | THC | CBG | CBC | CBN | CBDV | THCV |
| 2 | 57.85 | 36.84 | 1.31 | 3.38 | 0.11 | 0.25 | 0.26 |
| 3 | 58.19 | 36.41 | 1.60 | 3.43 | 0.03 | 0.13 | 0.20 |
| 6 | 59.63 | 33.70 | 2.56 | 3.31 | 0.10 | 0.32 | 0.38 |
| 7 | 61.26 | 32.45 | 2.09 | 3.55 | 0.10 | 0.29 | 0.26 |
| 8 | 65.73 | 26.39 | 3.23 | 3.89 | 0.13 | 0.33 | 0.30 |
| 9 | 63.19 | 30.34 | 2.32 | 3.43 | 0.12 | 0.29 | 0.32 |
| 10 | 64.87 | 28.31 | 2.63 | 3.38 | 0.11 | 0.36 | 0.32 |
| 11 | 61.30 | 32.73 | 2.09 | 3.23 | 0.08 | 0.30 | 0.27 |
| 12 | 62.97 | 29.44 | 3.31 | 3.55 | 0.09 | 0.33 | 0.32 |
| 13 | 64.10 | 30.18 | 1.89 | 3.03 | 0.13 | 0.35 | 0.32 |
| 14 | 60.58 | 32.14 | 3.46 | 3.05 | 0.12 | 0.32 | 0.32 |
| 15 | 61.69 | 32.36 | 2.00 | 3.18 | 0.06 | 0.33 | 0.38 |
| 16 | 53.02 | 40.73 | 2.80 | 2.85 | 0.07 | 0.26 | 0.27 |
| 17 | 63.49 | 30.14 | 2.37 | 3.35 | 0.08 | 0.31 | 0.26 |
| 18 | 69.95 | 23.07 | 2.22 | 3.83 | 0.08 | 0.42 | 0.42 |
| 19 | 61.91 | 30.63 | 2.94 | 3.84 | 0.08 | 0.32 | 0.28 |
| 20 | 65.38 | 27.59 | 2.37 | 3.84 | 0.08 | 0.33 | 0.41 |
| 21 | 69.04 | 25.39 | 1.29 | 3.74 | 0.10 | 0.12 | 0.33 |
| 22 | 68.60 | 25.55 | 1.18 | 4.15 | 0.11 | 0.12 | 0.29 |
| 23 | 66.41 | 26.58 | 2.46 | 4.10 | 0.12 | 0.12. | 0.21 |
| 24 | 69.14 | 25.30 | 1.17 | 3.92 | 0.09 | 0.13 | 0.25 |
| 25 | 62.02 | 32.34 | 1.64 | 3.58 | 0.10 | 0.11 | 0.21 |
| 26 | 61.51 | 32.33 | 1.09 | 4.63 | 0.08 | 0.15 | 0.20 |
| 27 | 54.67 | 39.41 | 2.15 | 3.14 | 0.06 | 0.32 | 0.26 |
| 28 | 63.04 | 31.95 | 0.91 | 3.29 | 0.18 | 0.30 | 0.34 |
| 29 | 61.80 | 32.10 | 1.68 | 3.67 | 0.16 | 0.29 | 0.30 |
| 30 | 56.84 | 37.65 | 1.73 | 2.95 | 0.16 | 0.31 | 0.37 |

Finally, as cannabis strains are often assessed and discussed in terms of their relative ratios of either major or minor cannabinoids, the CBD and THC to minor cannabinoid ratio (CBD+THC:minor cannabinoid) is described in Table 11.

TABLE 11
[CBD + THC]: minor cannabinoid ratio for CBD- and THC-enriched cannabis.

| | CBD + THC | CBD + THC: | CBD + THC: | CBD + THC: | CBD + THC: | CBD + THC: |
|---|---|---|---|---|---|---|
| Strain # | (mg/g) | CBG | CBC | CBN | CBDV | THCV |
| 2 | 87.29 | 72.14 | 27.98 | 872.90 | 379.52 | 363.71 |
| 3 | 148.62 | 58.98 | 27.57 | 2972.40 | 743.10 | 464.44 |
| 6 | 86.85 | 36.49 | 28.20 | 965.00 | 289.50 | 248.14 |
| 7 | 105.95 | 44.89 | 26.42 | 963.18 | 321.06 | 365.34 |
| 8 | 103.90 | 28.54 | 23.67 | 692.67 | 280.81 | 305.59 |
| 9 | 102.89 | 40.35 | 27.29 | 791.46 | 321.53 | 293.97 |
| 10 | 74.65 | 35.38 | 27.55 | 829.44 | 257.41 | 287.12 |
| 11 | 100.80 | 45.00 | 29.13 | 1120.00 | 315.00 | 347.59 |
| 12 | 104.01 | 27.96 | 26.07 | 1040.10 | 281.11 | 288.92 |
| 13 | 94.53 | 49.75 | 31.10 | 727.15 | 270.09 | 295.41 |
| 14 | 119.95 | 26.77 | 30.44 | 749.69 | 292.56 | 285.60 |
| 15 | 111.39 | 47.00 | 29.55 | 1591.29 | 285.62 | 247.53 |
| 16 | 171.45 | 33.49 | 32.84 | 1318.85 | 364.79 | 349.90 |
| 17 | 113.14 | 39.56 | 27.94 | 1131.40 | 305.78 | 364.97 |
| 18 | 89.86 | 41.99 | 24.29 | 1123.25 | 219.17 | 219.17 |
| 19 | 114.52 | 31.46 | 24.11 | 1145.20 | 293.64 | 327.20 |
| 20 | 122.65 | 39.19 | 24.19 | 1115.00 | 285.23 | 277.13 |
| 21 | 77.58 | 73.19 | 25.27 | 969.75 | 775.80 | 287.33 |
| 22 | 93.53 | 79.94 | 22.70 | 850.27 | 779.42 | 322.52 |
| 23 | 71.68 | 37.73 | 22.68 | 796.44 | 796.44 | 448.00 |
| 24 | 102.09 | 81.02 | 24.08 | 1020.90 | 729.21 | 378.11 |
| 25 | 112.47 | 57.68 | 26.34 | 937.25 | 865.15 | 449.88 |
| 26 | 125.80 | 86.16 | 20.26 | 1143.64 | 629.00 | 465.93 |
| 27 | 121.20 | 43.75 | 30.00 | 1515.00 | 295.61 | 367.27 |
| 28 | 98.54 | 104.83 | 28.90 | 518.63 | 317.87 | 281.54 |
| 29 | 65.49 | 55.97 | 25.58 | 595.36 | 327.45 | 311.86 |
| 30 | 71.18 | 54.75 | 32.06 | 593.17 | 309.48 | 254.21 |

### Cluster 3 Cannabinoid Profile

To fully describe the cannabinoid profile enriched for total THC and total CBG, quantitative analysis was performed on the 29 cannabis strains with a cannabinoid profile enriched for total THC and CBG. The results obtained from this analysis are provided in Table 12, below.

TABLE 12
Quantitative analysis of cannabinoids in THC- and CBG-enriched cannabis.

| | | | | | | | | Total |
|---|---|---|---|---|---|---|---|---|
| Cannabis | | | | | | | | cannabiniod |
| strain # | CBD | THC | CBG | CBC | CBN | CBDV | THCV | (mg/g) |
| 31 | 0.55 | 80.74 | 4.82 | 2.94 | 0.16 | 0 | 0.27 | 89.48 |
| 32 | 0.51 | 110.11 | 6.15 | 3.51 | 0.2 | 0 | 0.24 | 120.72 |
| 33 | 0.37 | 66.23 | 2.31 | 3.85 | 0.29 | 0 | 0.13 | 73.18 |
| 34 | 0.68 | 84.22 | 2.33 | 3.45 | 0.26 | 0 | 0.16 | 91.1 |
| 35 | 0.52 | 76.54 | 2.04 | 4.02 | 0.23 | 0 | 0.19 | 83.54 |
| 36 | 0.27 | 66.44 | 3.99 | 3.38 | 0.17 | 0 | 0.3 | 74.55 |
| 37 | 0.99 | 119.9 | 5.39 | 4.85 | 0.14 | 0 | 0.66 | 131.93 |
| 38 | 0.7 | 134.54 | 6.8 | 3.28 | 0.21 | 0 | 0.75 | 146.28 |
| 39 | 0.41 | 134.24 | 6.23 | 2.67 | 0.18 | 0 | 0.93 | 144.66 |
| 40 | 0.46 | 119.75 | 8.98 | 2.9 | 0.24 | 0 | 0.91 | 133.24 |
| 41 | 0.38 | 99.17 | 4.54 | 1.6 | 0.18 | 0 | 0.49 | 106.36 |
| 42 | 0.55 | 93.37 | 4.34 | 0.88 | 0.21 | 0 | 0.62 | 99.97 |
| 43 | 0.38 | 129.29 | 8.28 | 4.89 | 0.17 | 0 | 1.41 | 144.42 |
| 44 | 0.34 | 105.7 | 3.53 | 1.62 | 0.15 | 0 | 0.78 | 112.12 |
| 45 | 0.25 | 71.53 | 2.23 | 1.65 | 0.21 | 0 | 0.29 | 76.16 |
| 46 | 0.36 | 81.72 | 1.67 | 1.1 | 0.18 | 0 | 0.38 | 85.41 |
| 47 | 0.39 | 124.4 | 3.49 | 2.97 | 0.23 | 0 | 0.71 | 132.19 |
| 48 | 0.41 | 115.05 | 4.87 | 2.26 | 0.17 | 0 | 0.69 | 123.45 |
| 49 | 1.05 | 146.94 | 4.26 | 3.6 | 0.25 | 0 | 1.2 | 157.3 |
| 50 | 0.61 | 142.55 | 7.59 | 3.31 | 0.26 | 0 | 1.02 | 155.34 |
| 51 | 0.42 | 123.04 | 4.37 | 1.53 | 0.32 | 0 | 1.12 | 130.8 |
| 52 | 0.62 | 134.96 | 9.7 | 1.58 | 0.21 | 0 | 0.85 | 147.92 |
| 53 | 0.35 | 79.75 | 1.8 | 1.07 | 0.18 | 0 | 0.51 | 83.66 |
| 54 | 0.54 | 103.51 | 6.01 | 1.81 | 0.09 | 0 | 0.52 | 112.48 |
| 55 | 0.46 | 116.04 | 5.28 | 1.73 | 0.13 | 0 | 0.75 | 124.39 |
| 56 | 0.49 | 91.75 | 4.56 | 0.97 | 0.13 | 0 | 0.47 | 98.37 |
| 57 | 0.49 | 114.39 | 4.47 | 1.38 | 0.14 | 0 | 0.76 | 121.63 |
| 58 | 0.5 | 132.04 | 7.74 | 1.69 | 0.11 | 0 | 0.67 | 142.75 |
| 59 | 0.77 | 203.58 | 4.81 | 1.98 | 0.21 | 0 | 0.95 | 212.3 |

Using the total cannabinoid content (mg/g) for each of the analysed cannabis strains, the proportion of each cannabinoid in the total cannabinoid content of the plant material was derived to further characterise the cannabinoid profile of the cannabis strains, presented as a percentage (%) of the total cannabinoid content of the dry weight of plant material (Table 13).

TABLE 13
Major and minor cannabinoid content in THC- and CBG-enriched cannabis.

| Cannabis | | | | | | | |
|---|---|---|---|---|---|---|---|
| strain # | % CBD | % THC | % CBG | % CBC | % CBN | % CHDV | % THCV |
| 31 | 0.61 | 90.23 | 5.39 | 3.29 | 0.18 | 0.00 | 0.30 |
| 32 | 0.42 | 91.21 | 5.09 | 2.91 | 0.17 | 0.00 | 0.20 |
| 33 | 0.51 | 90.50 | 3.16 | 5.26 | 0.40 | 0.00 | 0.18 |
| 34 | 0.75 | 92.45 | 2.56 | 3.79 | 0.29 | 0.00 | 0.18 |
| 35 | 0.62 | 91.62 | 2.44 | 4.81 | 0.28 | 0.00 | 0.23 |
| 36 | 0.36 | 89.12 | 5.35 | 4.53 | 0.23 | 0.00 | 0.40 |
| 37 | 0.75 | 90.88 | 4.09 | 3.68 | 0.11 | 0.00 | 0.50 |
| 38 | 0.48 | 91.97 | 4.65 | 2.24 | 0.14 | 0.00 | 0.51 |
| 39 | 0.28 | 92.80 | 4.31 | 1.85 | 0.12 | 0.00 | 0.64 |
| 40 | 0.35 | 89.88 | 6.74 | 2.18 | 0.18 | 0.00 | 0.68 |
| 41 | 0.36 | 93.24 | 4.27 | 1.50 | 0.17 | 0.00 | 0.46 |
| 42 | 0.55 | 93.40 | 4.34 | 0.88 | 0.21 | 0.00 | 0.62 |
| 43 | 0.26 | 89.52 | 5.73 | 3.39 | 0.12 | 0.00 | 0.98 |
| 44 | 0.30 | 94.27 | 3.15 | 1.44 | 0.13 | 0.00 | 0.70 |
| 45 | 0.33 | 93.92 | 2.93 | 2.17 | 0.28 | 0.00 | 0.38 |
| 46 | 0.42 | 95.68 | 1.96 | 1.29 | 0.21 | 0.00 | 0.44 |
| 47 | 0.30 | 94.11 | 2.64 | 2.25 | 0.17 | 0.00 | 0.54 |
| 48 | 0.33 | 93.20 | 3.94 | 1.83 | 0.14 | 0.00 | 0.56 |
| 49 | 0.67 | 93.41 | 2.71 | 2.29 | 0.16 | 0.00 | 0.76 |
| 50 | 0.39 | 91.77 | 4.89 | 2.13 | 0.17 | 0.00 | 0.66 |
| 51 | 0.32 | 94.07 | 3.34 | 1.17 | 0.24 | 0.00 | 0.86 |
| 52 | 0.42 | 91.24 | 6.56 | 1.07 | 0.14 | 0.00 | 0.57 |
| 53 | 0.42 | 95.33 | 2.15 | 1.28 | 0.22 | 0.00 | 0.61 |
| 54 | 0.48 | 92.03 | 5.34 | 1.61 | 0.08 | 0.00 | 0.46 |
| 55 | 0.37 | 93.29 | 4.24 | 1.39 | 0.10 | 0.00 | 0.60 |
| 56 | 0.50 | 93.27 | 4.64 | 0.99 | 0.13 | 0.00 | 0.48 |
| 57 | 0.40 | 94.05 | 3.68 | 1.13 | 0.12 | 0.00 | 0.62 |
| 58 | 0.35 | 92.50 | 5.42 | 1.18 | 0.08 | 0.00 | 0.47 |
| 59 | 0.36 | 95.89 | 2.27 | 0.93 | 0.10 | 0.00 | 0.45 |

Finally, as cannabis strains are often assessed and discussed in terms of their relative ratios of either major or minor cannabinoids, the THC and CBG to minor cannabinoid ratio (THC+CBG:minor cannabinoid) is described in Table 14.

TABLE 14
THC + CBG: minor cannabinoid ratio for THC and CBG enriched cannabis.

| | Total | Ratio | Ratio | Ratio | Ratio | Ratio |
|---|---|---|---|---|---|---|
| Cannabis | THC + CBG: | THC + CBG: | THC + CBG: | THC + CBG: | THC + CBG: | THC + CBG: |
| strain # | (mg/g) | CBD | CBC | CBN | CBDV | THCV |
| 31 | 85.56 | 155.56 | 29.10 | 534.75 | NA | 316.89 |
| 32 | 116.26 | 227.96 | 33.12 | 581.30 | NA | 484.42 |
| 33 | 68.54 | 185.24 | 17.80 | 236.34 | NA | 527.23 |
| 34 | 86.55 | 127.28 | 25.09 | 332.88 | NA | 540.94 |
| 35 | 78.58 | 151.12 | 19.55 | 341.65 | NA | 413.58 |
| 36 | 70.43 | 260.85 | 20.84 | 414.29 | NA | 234.77 |
| 37 | 125.29 | 126.56 | 25.83 | 894.93 | NA | 189.83 |
| 38 | 141.34 | 201.91 | 43.09 | 673.05 | NA | 188.45 |
| 39 | 140.47 | 342.61 | 52.61 | 780.39 | NA | 151.04 |
| 40 | 128.73 | 279.85 | 44.39 | 536.38 | NA | 141.46 |
| 41 | 103.71 | 272.92 | 64.82 | 576.17 | NA | 211.65 |
| 42 | 97.71 | 177.65 | 111.03 | 465.29 | NA | 157.60 |
| 43 | 137.57 | 362.03 | 28.13 | 809.24 | NA | 97.57 |
| 44 | 109.23 | 321.26 | 67.43 | 728.20 | NA | 140.04 |
| 45 | 73.76 | 295.04 | 44.70 | 351.24 | NA | 254.34 |
| 46 | 83.39 | 231.64 | 75.81 | 463.28 | NA | 219.45 |
| 47 | 127.89 | 327.92 | 43.06 | 556.04 | NA | 180.13 |
| 48 | 119.92 | 292.49 | 53.06 | 705.41 | NA | 173.80 |
| 49 | 151.20 | 144.00 | 42.00 | 604.80 | NA | 126.00 |
| 50 | 150.14 | 246.13 | 45.36 | 577.46 | NA | 147.20 |
| 51 | 127.41 | 303.36 | 83.27 | 398.16 | NA | 113.76 |
| 52 | 144.66 | 233.32 | 91.56 | 688.86 | NA | 170.19 |
| 53 | 81.55 | 233.00 | 76.21 | 453.06 | NA | 159.90 |
| 54 | 109.52 | 202.81 | 60.51 | 1216.89 | NA | 210.62 |
| 55 | 121.32 | 263.74 | 70.13 | 933.23 | NA | 161.76 |
| 56 | 96.31 | 196.55 | 99.29 | 740.85 | NA | 204.91 |
| 57 | 118.86 | 242.57 | 86.13 | 849.00 | NA | 156.39 |
| 58 | 139.78 | 279.56 | 82.71 | 1270.73 | NA | 208.63 |
| 59 | 208.39 | 270.64 | 105.25 | 992.33 | NA | 219.36 |

### Cluster 4 Cannabinoid Profile

To fully describe the cannabinoid profile enriched for total THC, CBG and THCV, quantitative analysis was performed on the 12 cannabis strains with a cannabinoid profile enriched for total THC, CBG and THCV. The results obtained from this analysis are provided in Table 15, below.

TABLE 15
Quantitative analysis of cannabinoids in THC/CBG/THCV-enriched cannabis.

| | | | | | | | | Total |
|---|---|---|---|---|---|---|---|---|
| Cannabis | | | | | | | | cannabinoid |
| strain # | CBD | THC | CBG | CBC | CBN | CBDV | THCV | (mg/g) |
| 60 | 0.73 | 124.09 | 1.83 | 1.77 | 0.21 | 0.02. | 5.47 | 134.12 |
| 61 | 0.68 | 96.31 | 2.74 | 2.29 | 0.3 | 0.02 | 4.54 | 106.88 |
| 62 | 0.46 | 120.87 | 5.3 | 2.13 | 0.24 | 0.02 | 3.26 | 132.28 |
| 63 | 0.8 | 104.57 | 8.89 | 1.94 | 0.18 | 0.03 | 6.57 | 122.98 |
| 64 | 0.42 | 139.17 | 4.47 | 2.32 | 0.2 | 0.02 | 5.63 | 152.23 |
| 65 | 0.29 | 85.67 | 1.47 | 1.21 | 0.12 | 0.02. | 2.74 | 91.52 |
| 66 | 0.66 | 99.73 | 1.37 | 1.32 | 0.12 | 0.02 | 2.79 | 106.01 |
| 67 | 0.61 | 86.69 | 1.5 | 0.99 | 0.14 | 0.02 | 3.12 | 93.07 |
| 68 | 0.38 | 96.7 | 2.08 | 5.74 | 0.25 | 0.02 | 2.68 | 107.85 |
| 69 | 0.5 | 81.06 | 3.08 | 1.58 | 0.22 | 0.02 | 2.3 | 88.76 |
| 70 | 0.27 | 78.86 | 1.5 | 3.23 | 0.3 | 0.02 | 2.23 | 86.41 |
| 71 | 0.45 | 83.39 | 2.01 | 1.97 | 0.26 | 0.02 | 2.49 | 90.59 |

Using the total cannabinoid content (mg/g) for each of the analysed cannabis strains, the proportion of each cannabinoid in the total cannabinoid content of the plant material was derived to further characterise the cannabinoid profile of the cannabis strains, presented as a percentage (%) of the total cannabinoid content of the dry weight of plant material (Table 16).

TABLE 16
Major and minor cannabinoid content in
THC/CBG/THCV-enriched cannabis.

| Cannabis | % | % | % | % | % | % | % |
|---|---|---|---|---|---|---|---|
| strain # | CBD | THC | CBG | CRC | CBN | CBDV | THCV |
| 60 | 0.54 | 97.52 | 1.36 | 1.32 | 0.16 | 0.01 | 4.08 |
| 61 | 0.64 | 90.11 | 2.56 | 2.14 | 0.28 | 0.02 | 4.25 |
| 62 | 0.35 | 91.37 | 4.01 | 1.61 | 0.18 | 0.02 | 2.46 |
| 63 | 0.65 | 85.03 | 7.23 | 1.58 | 0.15 | 0.02 | 5.34 |
| 64 | 0.28 | 91.42 | 2.94 | 1.52 | 0.13 | 0.01 | 3.70 |
| 65 | 0.32 | 93.61 | 1.61 | 1.32 | 0.13 | 0.02 | 2.99 |
| 66 | 0.62 | 94.08 | 1.29 | 1.25 | 0.11 | 0.02 | 2.63 |
| 67 | 0.66 | 93.14 | 1.61 | 1.06 | 0.15 | 0.02 | 3.35 |
| 68 | 0.35 | 89.66 | 1.93 | 5.32 | 0.23 | 0.02 | 2.48 |
| 69 | 0.56 | 91.32 | 3.47 | 1.78 | 0.25 | 0.02 | 2.59 |
| 70 | 0.31 | 91.26 | 1.74 | 3.74 | 0.35 | 0.02 | 2.58 |
| 71 | 0.50 | 92.05 | 7.72 | 2.17 | 0.29 | 0.02 | 2.75 |

Finally, as cannabis strains are often assessed and discussed in terms of their relative ratios of either major or minor cannabinoids, the THC to minor cannabinoid ratio (THC:minor cannabinoid) is described in Table 17.

TABLE 17
THC: minor cannabinoid ratio for THC/CBG/THCV enriched cannabis.

| | Ratio | Ratio | Ratio | Ratio | Ratio | Ratio |
|---|---|---|---|---|---|---|
| Cannabis | THC: | THC: | THC: | THC: | THC: | THC: |
| strain # | CBG | THCV | CBD | CBC | CBN | CBDV |
| 60 | 67.81 | 22.69 | 169.99 | 70.11 | 590.90 | 6204.50 |
| 61 | 35.15 | 21.21 | 141.63 | 42.06 | 321.03 | 4815.50 |
| 62 | 22.81 | 37.08 | 262.76 | 56.75 | 503.63 | 6043.50 |
| 63 | 11.76 | 15.92 | 130.71 | 53.90 | 580.94 | 3485.67 |
| 64 | 31.13 | 24.72 | 331.36 | 59.99 | 695.85 | 6958.50 |
| 65 | 58.28 | 31.27 | 295.41 | 70.80 | 713.92 | 4283.50 |
| 66 | 72.80 | 35.75 | 151.11 | 75.55 | 831.08 | 4986.50 |
| 67 | 57.79 | 27.79 | 142.11 | 87.57 | 619.21 | 4334.50 |
| 68 | 46.49 | 36.08 | 254.47 | 16.85 | 386.80 | 4835.00 |
| 69 | 26.32 | 35.24 | 162.12 | 51.30 | 368.45 | 4053.00 |
| 70 | 52.57 | 35.36 | 292.07 | 24.41 | 262.87 | 3943.00 |
| 71 | 41.49 | 33.49 | 185.31 | 42.33 | 320.73 | 4169.50 |

### C. Quantitative Terpene Analysis

### Reagents and Standards

Individual authentic standards and standard solutions for compound identification and were obtained from Sigma-Aldrich (Ryde, NSW, Australia) and Leco Australia (Baulkham Hills, NSW, Australia) as distributer for Restek (Bellefonte, Calif. USA). Combined Cannabis Terpenes standard solutions #1 and #2 (CT, 2.5 mg/mL in isopropanol) were purchased from Restek. Dodecane (internal standard) and C₈-C₂₀ alkane mix were obtained from Sigma. For compound identification, individual standards were diluted in Hexane (Sigma standards) or Methanol (Restek individual standards) to between 20-100 ng/μL. The combined CT standards were used for quantitation, spiking and LOD/LOQ experiments. Dodecane and sabinene stock solutions were prepared at 10 mg/mL. CT standards #1 and #2 and sabinene stock solution were combined in hexane to obtain a solution of 100 μg/mL (Table 18). This solution was then serially diluted 1:2 in hexane nine more times to obtain a lowest concentration of 0.195 μg/mL. To these, internal standard was added to a final concentration of 50 μg/mL.

TABLE 18
Terpenes standards used to identify and quantitate terpenes.

| Name | m/z | RT (min) | Source* |
|---|---|---|---|
| α-Pinene (+/−) | 93.0 | 8.056 | CT1 |
| Camphene | 93.0 | 8.505 | CT1 |
| Sabinene | 93.0 | 9.067 | Ind. |
| β-Pinene (+/−) | 93.0 | 9.215 | CT1 |
| Myrcene | 93.0 | 9.380 | CT1 |
| 3-Carene | 93.0 | 10.007 | CT1 |
| α-Terpinene | 93.0 | 10.230 | CT1 |
| p-Cymene | 119.0 | 10.437 | CT1 |
| Limonene | 68.1 | 10.565 | CT1 |
| Eucalyptol | 81.0 | 10.678 | CT2 |
| Ocimene isomer | 93.1 | 10.960 | CT1 |
| γ-Terpinene | 93.1 | 11.367 | CT1 |
| Terpinolene | 93.1 | 12.150 | CT1 |
| Linalool | 93.0 | 12.496 | CT1 |
| Isopulegol | 121.0 | 14.022 | CT1 |
| α-Terpineol | 93.1 | 15.368 | CT1 |
| Nerolidol | 69.1 | 16.783 | CT1 |
| trans-Caryophyllene | 93.0 | 21.600 | CT1 |
| Humulene | 93.0 | 22.524 | CT1 |
| β-cis-Caryophyllene | 69.2 | 24.968 | CT1 |
| Caryophyllene Oxide | 79.0 | 25.691 | CT2 |
| Guaiol | 105.1 | 25.970 | CT1 |
| a-Bisabolol | 93.0 | 27.989 | CT1 |

*CT1: Resteck Cannabis Terpenes Standard mix #1 CT2: Resteck Cannabis Terpenes Standard mix #2, ind: individual standard (Sigma-Aldrich).

### Sample Preparation

Dried and ground plant material was obtained from the Victorian Government Medicinal Cannabis Cultivation Facility. Chemically diverse strains were selected as described above at [0180] and combined to create a test sample that represented the largest possible variety of volatile compounds.

40 mg of combined sample were weighed into 2 mL microcentrifuge tubes and 400 μL of extraction solvent (50 mg/L dodecane in hexane) were added. Samples were vortexed for 30 seconds, then sonicated for 10 minutes, and centrifuged at maximum g for 5 minutes. The supernatant was removed to an autosampler vial, the extraction procedure was repeated and first and second extracts combined. Hexane, isopropanol and ethyl acetate were evaluated as extraction solvents, and ultimately, hexane was chosen for the validation.

For measuring myrcene concentration, samples were further diluted 1:5 in hexane to bring the analyte concentration into the linear range.

### Gas Chromatography/Mass Spectroscopy (GC-MS) Analysis

Samples were analysed using an Agilent 7000 GC-QQQ system equipped with EPC backflush option and a Gerstal MultiPurpose Sampler MPS. Separation was carried out using an Agilent (Santa Clara, USA) J&W DB-5MS-DG column, 30 m×0.25 mm I.D,×0.25 μm film thickness with an integral 10 m guard column, connected to a 2.5 m×0.18 mm deactivated fused silica capillary (non-coated) via a purged union.

TABLE 19
Liquid Extraction and GC-MC parameters used for
quantitative determination of terpenes from cannabis.
GC-MS parameters for liquid injection analysis

| Sample | 40 mg milled, dried cannabis bud, extracted twice with 400 μL solvent. | |
|---|---|---|
| | Extracts combined for analysis | |
| Oven | Time (min) | Temperature (° C.) |
| | 0 | 60 |
| | 2 | 60 |
| | 30 | 200 |
| | 31 | 320 (post run) |
| | 34 | 320 (post run) |
| | TOTAL RUN TIME | 30 min |
| | POST RUN TIME | 5 min |

| | |
|---|---|
| | Helium, Flow: 1.38 mL/min for column 1 and constant flow of 1.5 mL/min |
| | for column 2 via purged union |
| Injector | 1:20 split, 270° C., 2 μL |
| Flow rates and | Main Run |
| Backflush | Column 1: flow 1.3801 mL/min; average velocity 31.917 cm/sec; |
| settings | Column 2: flow 1.5 mL/min; 0.87846 psi; average velocity 155.4 cm/sec |
| | Backflush |
| | Column 1: 5 min (post-run) −1.6778 mL/min; |
| | [negative flow, going out the inlet] |
| | Column 2: 5 min (post-run) 5.9911 mL/min. |
| | [positive flow, going into MS]. |

### Data Processing

Compounds were putatively identified by spectral matching against the NIST 2008 library and retention time indices in the NIST webbook using the method of Van den Dool and Kratz (1963, Journal of Chromatography, 11: 463-471). Predictions were confirmed using authentic standards where available. Quantitation was performed using Agilent MassHunter Quant software with the largest fragment ion (base peak) used for quantitation.

### Validation Parameters

The method was validated for resolution, linearity, quantitation and detection limits, accuracy and precision for the compounds listed in Table 19. Resolution (R) for two adjacent peaks was determined using extracted ion chromatograms for the base peak ion (quantifier) of each detected compound, based on their retention times T1/T2 and peak widths w1/w2:

R_1,2=2(T_2−T_1)/(w_2+w_1)

For determining linearity, LOD and LOQ, a combined CT and sabinene standard was injected at ten concentrations from 100 μg/mL to 0.195 ng/mL, based on a 1:2 dilution series of the 100 μg/mL standard. Calibration curves were required to have R² values greater than or equal to 0.99. LOD and LOQ were determined as LOD=3.3*σ/S and LOQ=10*σ/S, with a representing the standard deviation of the compound and S the slope of the compounds calibration curve.

To determine method accuracy, three replicates of a sample preparation were spiked at 25, 12.5 and 2.5 μg/mL CT and sabinene (equivalent to 0.5, 0.25 and 0.05 mg/g) and extracted as described above. Unspiked plant material was extracted to determine endogenous levels. Recoveries were determined as (measured concentration−endogenous concentration)/spiked concentration×100. Acceptable recoveries were defined as 80-120%

Precision and intermediate precision were determined by analysing eight individual preparations of plant material extracted independently by two analysts on different days. The relative standard deviation for the concentration of the compounds was to be 56% for individual analysts and ≤8% combined.

### Results

### Solvent Optimisation

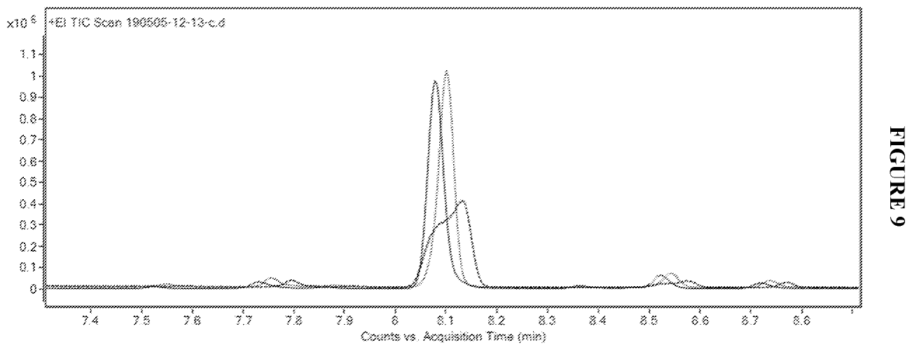

Three solvents were initially trialled for liquid extraction: hexane, isopropanol and ethyl acetate. All three performed equally well in terms of extraction efficiency, but both isopropanol and to a lesser extend ethyl acetate caused early eluting compounds to show undesirable peak shapes during GC analysis. A comparison of peak shapes is shown in FIG. 9.

Compounds dissolved in hexane produced the best peak shape. In view of these data, hexane was used for further method validation.

### Validation Results

### Compound Identification and Resolution

A total of 49 individual compounds were detected across multiple strains used, 25 of those are monoterpenes and monoterpenoids, the remainder are sesquiterpenes and sesquiterpenoids. Terpene names, base peak (quantifier) ion m/z, retention times and indices, resolution factors and identification status are summarised in Table 20. Most monoterpene identities predicted by spectral library matching were confirmed using authentic standards, with the exception of an unknown Monoterpene (Monoterpene 01), fenchol and trans-2-pinanol. β-phellandrene and thujanol identities were confirmed by matching published retention times indices.

TABLE 20
Compounds detected in this study.

| Name | m/z | RT | RI | Rp | Rf | Status |
|---|---|---|---|---|---|---|
| Monoterpene01 | 93 | 7.866 | 932 | n/a | 1.7 | specID |
| α-Pinene (+/−) | 93 | 8.056 | 940 | 1.9 | 1.6 | Val |
| Camphene | 93 | 8.505 | 958 | 4.8 | 1.7 | Val |
| Sabinene | 68.1 | 9.067 | 979 | 1.5 | 1.2 | Val |
| β-Pinene (+/−) | 69.2 | 9.215 | 985 | 1.3 | 1.4 | Val |
| Myrcene | 93 | 9.380 | 991 | 2.6 | 1.4 | Val |
| 3-Carene | 79 | 10.007 | 1014 | 1.9 | 1.6 | Val |
| α-Terpinene | 119 | 10.230 | 1023 | 2.0 | 1.6 | Val |
| p-Cymene | 93 | 10.437 | 1031 | 4.1 | 1.9 | Val |
| Limonene | 121 | 10.565 | 1035 | 3.0 | 15.7 | Val |
| β-Phellandrene | 93.1 | 10.660 | 1039 | 1.2 | 1 | specID, RI |
| Eucalyptol | 93 | 10.678 | 1040 | 1.7 | 2.1 | Val |
| Ocimene isomer | 105.1 | 10.960 | 1050 | 1.8 | 2.2 | Val |
| γ-Terpinene | 93.1 | 11.367 | 1064 | 2.9 | 2.3 | Val |
| 4-Thujanol | 93.1 | 11.750 | 1077 | 3.4 | 2.9 | specID, RI |
| Terpinolene | 93 | 12.150 | 1090 | 1.7 | 2.5 | Val |
| Fenchone | 81.1 | 12.333 | 1096 | 1.8 | 1.7 | confirmed |
| Linalool | 81 | 12.496 | 1101 | 1.4 | 2.7 | Val |
| Fenchol | 81.1 | 13.201 | 1128 | 7.4 | 2.0 | specID |
| trans-2-Pinanol | 93.1 | 13.421 | 1135 | 2.0 | 13 | specID |
| Isopulegol | 93 | 14.022 | 1156 | 1.6 | 2.8 | Val |
| Borneol | 95.1 | 14.758 | 1181 | 5.4 | 4.5 | confirmed |
| α-Terpineol | 93.1 | 15.368 | 1201 | 3.4 | 2.9 | Val |
| Dodecane | 57.1 | 15.370 | 1201 | 6.7 | 7.2 | IS |
| Nerolidol | 93.1 | 16.783 | 1253 | 2.6 | 22.5 | Val |
| β-Bergamotene | 119.1 | 21.015 | 1406 | 3.0 | 3.4 | specID |
| α-Bergamotene | 93.1 | 21.341 | 1420 | 3.6 | 2.3 | specID |
| trans-Caryophyllene | 93.1 | 21.600 | 1430 | 2.9 | 3.4 | Val |
| γ-Elemene iso1 | 121 | 21.768 | 1437 | 1.4 | 1.8 | specID |
| Bergamotene iso3 | 119 | 21.848 | 1440 | 0.8 | 3.5 | specID, RI |
| α-Guaiene | 105 | 21.944 | 1444 | 1.7 | 1.8 | specID |
| Farnesene | 69.2 | 22.270 | 1456 | 4.1 | 2.1 | confirmed |
| Humulene | 69.1 | 22.524 | 1466 | 3.9 | 32.1 | Val |
| epi-β-Selinene | 93.1 | 23.396 | 1499 | 1.4 | 1.5 | specID |
| (-)-α-Selinene | 105.1 | 23.567 | 1506 | 1.7 | 1.1 | specID, RI |
| Sesquiterpenes, coeluting | 93.1 | 23.574 | 1506 | 1.5 | 1.4 | specID |
| δ-Guaiene | 161 | 23.662 | 1510 | 1.4 | 2.6 | specID, RI |
| β-Guaiene | 161 | 24.139 | 1530 | 1.2 | 1.5 | speciD |
| Farnesol | 69 | 24.222 | 1534 | 3.2 | 3.7 | confirmed |
| α-Bisabolene | 93.1 | 24.512 | 1546 | 1.1 | 0.7 | specID |
| Guaia-3,9-diene | 161.1 | 24.572 | 1548 | 1.5 | 1.6 | specID |
| 3,7(11)-Selinadiene | 161.1 | 24.680 | 1553 | 1.6 | 1.1 | specID |
| β-cis-Caryophyllene | 93 | 24.968 | 1564 | 2.5 | 4.1 | Val |
| γ-Elemene iso2 | 121.1 | 25.152 | 1572 | 1.4 | 2.8 | specID |
| Caryophyllene Oxide | 93 | 25.691 | 1593 | 1.6 | 4.8 | Val |
| Guaiol | 93 | 25.970 | 1605 | 2.3 | 7.0 | Val |
| β-Cadinene | 189.1 | 26.666 | 1636 | 1.9 | 1.3 | specID |
| γ-Gurjunene | 59.1 | 27.400 | 1667 | 1.8 | 1.5 | specID |
| sesquiterpene | 107 | 27.578 | 1675 | 1.2 | 1.4 | specID |
| α-Bisabolol | 93 | 27.989 | 1692 | 1.9 | n/a | Val |

*Base peak (quantifier) ions (m/z), retention times (RT) and indices (RI), resolution to the closest preceding (Rp) and following peaks (Rf) and identification status (specID): based on library match, RI: based and library match and retention time index; confirmed: matches spectrum and RT of authentic standard; Val: confirmed compound used for method validation).

### Linearity

A combined CT and sabinene standard was injected at ten concentrations from 100 μg/mL to 0.195 ng/mL, based on a 1:2 dilution series of the 100 μg/mL standard. All calibration curves obtained were linear with R² values greater than or equal to 0.994. Based on individual detection limits, the curves span all ten calibration points, except linalool, ocimene and trans-caryophyllene (100-0.391 μg/mL), caryophyllene oxide, guaiol and isopulegol, (100-1.56 μg/mL) and α-bisabolol and β-cis-caryophyllene (100-12.5 μg/mL). Nerolidol was removed from the analysis due to poor peak shapes and high detection limits.

### Detection and Quantitation Limits

Detection limits (LOD) range from 0.017 μg/mL to 1.144 μg/mL, Quantitation limits (LOQ) range from 0.052 to 3.468 μg/mL (with β-pinene and α-bisabolol having the lowest and highest values, respectively, for both LOD/LOQ); Table 21.

TABLE 21
Detection (LOD) and Quantitation (LOQ) limits for the validation compounds.

| Compound | Slope of calibration curve | LOD μg/mL | LOQ μg/mL |
|---|---|---|---|
| α-Pinene (+/−) | 0.039129 | 0.022 | 0.068 |
| Camphene | 0.023849 | 0.032 | 0.097 |
| Sabinene | 0.038856 | 0.026 | 0.079 |
| β-Pinene (+/−) | 0.047192 | 0.017 | 0.052 |
| Myrcene | 0.028807 | 0.047 | 0.142 |
| 3-Carene | 0.030862 | 0.030 | 0.091 |
| α-Terpinene | 0.021459 | 0.038 | 0.115 |
| p-Cymene | 0.078714 | 0.021 | 0.065 |
| Limonene | 0.017349 | 0.066 | 0.200 |
| Eucalyptol | 0.008849 | 0.115 | 0.348 |
| Ocimene | 0.015782 | 0.057 | 0.174 |
| γ-Terpinene | 0.032899 | 0.046 | 0.139 |
| Terpinolene | 0.018898 | 0.045 | 0.137 |
| Linalool | 0.013307 | 0.060 | 0.181 |
| Isopulegol | 0.005376 | 0.045 | 0.136 |
| trans-Caryophyllene | 0.010502 | 0.092 | 0.326 |
| Humulene | 0.032275 | 0.021 | 0.065 |
| β-cis-Caryophyllene | 0.005202 | 0.388 | 1.177 |
| Caryophyllene Oxide | 0.004599 | 0.252 | 0.764 |
| Guaiol | 0.008546 | 0.107 | 0.324 |
| α-Bisabolol | 0.010087 | 1.144 | 3.468 |

### Accuracy

To determine method accuracy, three replicates of a sample preparation were spiked with 0.5, 0.25 and 0.05 mg/g CT and sabinene and extracted as previously. Unspiked plant material was extracted to determine endogenous levels. The recoveries of the spike as expressed in % adjusted for endogenous levels ranged from 121.4 to 61.8%, with the majority of compounds ranging between 80-120% recovery across the three concentrations (see Table 22, below).

TABLE 22
Recovery of analytes in spiked sample
at three concentration levels.

| Compound | Spike recovery (%) | | |
|---|---|---|---|
| Level | High | Mid | Low |
| α-Pinene (+/−) | 106.4 | 108.2 | 97.5 |
| Camphene | 112.6 | 121.4 | 119.0 |
| Sabinene | 108.8 | 115.7 | 98.7 |
| β-Pinene (+/−) | 110.7 | 113.7 | 102.2 |
| Myrcene | 106.5 | 104.8 | 95.5 |
| 3-Carene | 112.4 | 118.7 | 115.7 |
| α-Terpinene | 78.8 | 84.5 | 81.8 |
| p-Cymene | 112.2 | 115.6 | 116.5 |
| Limonene | 110.6 | 114.6 | 107.9 |
| Eucalyptol | 116.6 | 109.9 | 119.2 |
| Ocimene | 101 | 105.7 | 98.6 |
| γ-Terpinene | 104.6 | 108.9 | 101.6 |
| Terpinolene | 97.1 | 100.4 | 93.0 |
| Linalool | 113.6 | 114.5 | 105.5 |
| Isopulegol | 61.8 | 66.2 | 82.6 |
| trans-Caryophyllene | 93.5 | 99.1 | 89.7 |
| Humulene | 92.5 | 98.3 | 90.7 |
| β-cis-Caryophyllene | 62.0 | 72.9 | 87.7 |
| Caryophyllene Oxide | 69.0 | 76.3 | 84.8 |
| Guaiol | 102.6 | 104.1 | 93.3 |
| α-Bisabolol | 98.7 | 100.3 | 93.5 |

### Precision and Intermediate Precision

Eight individual preparations of plant material were extracted independently by two analysts on different days and analysed separately. % RSDs varied from 1.13 to 8.74 for single analysts, 1.65 to 7.54 for both analysts combined. Three compounds, α-terpinene, eucalyptol and terpinolene were found to have % RSDs>10. Results are summarised in Table 23. Myrcene content was determined from a 1:5 dilution of the original extract to bring the concentration back into the linear range.

TABLE 23
Precision and intermediate precision results.

| | A1 | A1 | A2 | A2 | combined | combined |
|---|---|---|---|---|---|---|
| | Average | % RSD | Average | % RSD | average | % RSD |
| α-Pinene (+/−) | 0.132 | 8.74 | 0.123 | 3.48 | 0.127 | 7.54 |
| Camphene | 0.035 | 8.27 | 0.033 | 3.61 | 0.034 | 7.01 |
| Sabinene | 0.005 | 5.07 | 0.005 | 2.54 | 0.005 | 3.92 |
| β-Pinene +/−) | 0.233 | 7.56 | 0.221 | 2.95 | 0.227 | 6.17 |
| Myrcene (1:5) | 1.38 | 2.07 | 1.318 | 4.43 | 1.349 | 4.92 |
| 3-Carene | n/d | | | | | |
| α-Terpinene | 0.005 | 16.81 | 0.005 | 7.86 | 0.005 | 13.86 |
| p-Cymene | n/d | | | | | |
| Limonene | 1.753 | 5.74 | 1.744 | 3.4 | 1.749 | 4.62 |
| Eucalyptol | 0.027 | 20.86 | 0.028 | 13.55 | 0.028 | 18.68 |
| Ocimene | n/d | | | | | |
| γ-Terpinene | 0.006 | 4.87 | 0.006 | 4.38 | 0.006 | 5.61 |
| Terpinolene | 0.015 | 10.26 | 0.014 | 3.73 | 0.014 | 7.23 |
| Linalool | 0.017 | 2.77 | 0.018 | 2.75 | 0.017 | 4.46 |
| Isopulegol | 0.06 | 2.43 | 0.06 | 1.97 | 0.06 | 2.52 |
| Nerolidol | n/d | | | | | |
| trans-Caryophyllene | 1.492 | 1.76 | 1.449 | 1.93 | 1.47 | 2.31 |
| Humulene | 0.419 | 2.44 | 0.408 | 1.66 | 0.413 | 2.6 |
| β-cis-Caryophyllene | 0.198 | 1.44 | 0.201 | 1.9 | 0.2 | 1.65 |
| Caryophyllene Oxide | 0.121 | 2.79 | 0.129 | 2.11 | 0.125 | 3.8 |
| Guaiol | 0.479 | 1.86 | 0.491 | 2.13 | 0.485 | 2.57 |
| α-Bisabolol | 0.553 | 1.13 | 0.565 | 1.65 | 0.559 | 2.62 |

*Averages are expressed as μg/g dry biomass. Compounds not detected in the material used are marked as n/d.

Our initial relative quantitation and identification work was carried out using static headspace as described above, and the method reliably provided very strong signals for all monoterpenes and earlier eluting sesquiterpenes. Liquid extraction methods are also robust for absolute quantitation.

### D. Conclusion

The optimised GC-MS analysis methods described herein allows the determination of relative terpene content in cannabis by static headspace, SPME or liquid injection. Of the three columns tested the best resolution was obtained with the DB5 column. Static headspace was effective for the analysis of monoterpenes, while sesquiterpenes were more effectively extracted using a hexane-based liquid approach. PCA analysis of 67 cannabis strains proved that there was significant diversity in this population, although in most cases myrcene was the most abundant terpene present. This diversity will allow the selection of medicinal cannabis strains based not only on their cannabinoid content but also their terpene profile.

As described herein, liquid extraction with hexane is an effective extraction method for absolute quantitation of both monoterpenes and sesquiterpenes. This finding has been reduced to practice in a quantitative method, which is capable of measuring terpenes (i.e., monoterpenes and sesquiterpenes) in cannabis plant material that is particularly useful in the identification and selection of cannabis plants with terpene profiles that are beneficial for therapeutic use.

1-27. (canceled)

28. A method for determining a monoterpene profile of cannabis plant material, the method comprising:
a. obtaining a reference spectrum of known monoterpenes;
b. selectively extracting one or more monoterpenes from the cannabis plant material, preferably wherein the plant material comprises one or both of inflorescence and leaf, preferably wherein the plant material is derived from a female cannabis plant;
c. obtaining spectroscopic data from the monoterpenes separated in step (b);
d. comparing the spectroscopic data to the reference spectrum to identify and/or measure the level of one or more monoterpenes to determine the monoterpene profile of the cannabis plant material.

29. The method of claim 28, wherein the selective extraction of one or more monoterpenes according to step (a) is performed using an extraction method selected from the group consisting of headspace extraction and hexane-based liquid extraction.

30. The method of claim 29, wherein the headspace extraction method is selected from the group consisting of headspace solid phase micro-extraction (SPME) and static headspace extraction; preferably wherein the headspace extraction method is static headspace extraction.

31. The method of claim 28, wherein the one or more monoterpenes are selected from the group consisting of α-phellandrene, α-pinene, camphene, β-pinene, myrcene, limonene, eucalyptol, γ-terpinene and linalool.

32. The method of claim 31, wherein the one or more monoterpenes are selected from the group consisting of α-pinene, β-pinene, myrcene, limonene and linalool, preferably wherein the one or more monoterpenes are selected from the group consisting of β-pinene and myrcene.

33. The method of claim 28, wherein the spectroscopic data are measured by gas chromatography-mass spectrometry (GC-MS).

34. The method of claim 33, wherein the gas chromatography is performed using a DB-5 capillary GC column.

35. A method for determining a sesquiterpene profile of cannabis plant material, the method comprising:
a. obtaining a reference spectrum of known sesquiterpenes;
b. selectively extracting one or more sesquiterpenes from the cannabis plant material, preferably wherein the plant material comprises one or both of inflorescence and leaf, preferably wherein the plant material is derived from a female cannabis plant;
c. obtaining spectroscopic data from the sesquiterpenes separated in step (b);
d. comparing the spectroscopic data to the reference spectrum to identify and/or measure the level of one or more sesquiterpenes to determine the sesquiterpene profile of the cannabis plant material.

36. The method of claim 35, wherein the selective extraction of one or more sesquiterpenes components according to step (a) is performed by liquid extraction, preferably wherein the liquid extraction is a hexane-based liquid extraction.

37. The method of claim 35, wherein the one or more sesquiterpenes is selected from the group consisting of γ-elemene, humulene, nerolidol, guaia-3,9-diene and caryophyllene, preferably wherein the sesquiterpene is caryophyllene.

38. The method of claim 35, wherein the spectroscopic data are measured by gas chromatography-mass spectrometry (GC-MS).

39. The method of claim 38, wherein the gas chromatography is performed using a DB-5 capillary GC column.

40. The method of claim 28, wherein the cannabis plant material has a predetermined cannabinoid profile that is selected from the group consisting of: (i) a cluster 1 cannabinoid profile; (ii) a cluster 2 cannabinoid profile; (iii) a cluster 3 cannabinoid profile; and (iv) a cluster 4 cannabinoid profile.

41. The method of claim 28, further comprising:
e. repeating steps (a)-(d) on at least particularly dried plant material harvested from a plurality of different cannabis plants; and
f. on the basis of the measurements from step (e), selecting from the plurality of different cannabis plants a cannabis plant comprising a terpene profile that is enriched for monoterpenes.

42. The method of claim 35, further comprising:
e. repeating steps (a)-(d) on at least partially dried plant material harvested from a plurality of different cannabis plants; and
f. on the basis of the measurements from step (e), selecting from the plurality of different cannabis plants a cannabis plant comprising a terpene profile that is enriched for sesquiterpene.

43. The method of claim 41, wherein the cannabis plant selected from step (d) further comprises a predetermined cannabinoid profile selected from the group consisting of: (i) a cluster 1 cannabinoid profile; and (ii) a cluster 2 cannabinoid profile.

44. The method of claim 35, wherein the cannabis plant material has a predetermined cannabinoid profile that is selected from the group consisting of: (i) a cluster 1 cannabinoid profile; (ii) a cluster 2 cannabinoid profile; (iii) a cluster 3 cannabinoid profile; and (iv) a cluster 4 cannabinoid profile.

45. The method of claim 42, wherein the cannabis plant selected from step (d) further comprises a predetermined cannabinoid profile selected from the group consisting of: (i) a cluster 1 cannabinoid profile; and (ii) a cluster 2 cannabinoid profile.
